(19) United States
(12) Patent Application Publication
(10) Pub. No.: US 2026/0077201 A1
(43) Pub. Date: Mar. 19, 2026

(54) Spinal Cord Stimulation Device

(71) Applicant: Reach Neuro, Inc., Pittsburgh, PA (US)

(72) Inventors: Marc Philip Powell, Pittsburgh, PA (US); Camila Pagán Santiago, Pittsburgh, PA (US); Charli Ann Hooper, Pittsburgh, PA (US)

(21) Appl. No.: 19/104,936

(22) PCT Filed: Aug. 18, 2023
(86) PCT No.: PCT/US2023/030624

§ 371 Date: Feb. 19, 2025

Related U.S. Application Data
(60) Provisional application No. 63/399,457, filed on Aug. 19, 2022.

(51) Int. Cl. A61N 1/36; A61N 1/05; A61N 1/375

(52) U.S. Cl. CPC A61N 1/36139; A61N 1/0551; A61N 1/36062; A61N 1/36125; A61N 1/37518

(57) ABSTRACT

The present disclosure describes methods, sensors, and systems for detecting movement of a spine and stimulating specific regions of the spinal cord in response to the movement. The disclosure includes sensors having one or more sensing elements to sense movement of the spine and one or more stimulating elements configured to electrically stimulate an area proximate the spine. When a movement of the spine is detected via the sensor, the one or more stimulating elements modulate an electrical stimulus.

### CROSS REFERENCES

This application is a national phase entry under 35 U.S.C. § 371 of International Patent Application PCT/US2023/030624, filed Aug. 18, 2023, designating the United States of America and published in English as International Patent Publication WO2024/039877 on Feb. 22, 2024, which claims the benefit under Article 8 of the Patent Cooperation Treaty to U.S. Provisional Patent Application No. 63/399,457 entitled “Spinal Cord Stimulation Device” filed on Aug. 19, 2022.

### TECHNICAL FIELD

The present disclosure generally relates to a spinal cord stimulation device, and more particularly, a spinal cord stimulation device and sensor for detecting movement of the spine and providing stimulation to the spinal cord.

### BRIEF DESCRIPTION OF THE DRAWINGS

The following detailed description of embodiments of the spinal cord stimulation device and spinal lead will be better understood when read in conjunction with the appended drawings of exemplary embodiments. It should be understood, however, that the disclosure is not limited to the precise arrangements and instrumentalities shown.

FIG. 1 is an illustration of a spinal cord stimulation device having an electrode/sensor in accordance with an exemplary embodiment of the present disclosure;

FIG. 2A is a top plan view of the sensor of FIG. 1 having a plurality of sensing elements aligned in a first direction in accordance with an exemplary embodiment of the present disclosure;

FIG. 2B is a top plan view of the sensor of FIG. 2A having a plurality of sensing elements aligned in a second direction;

FIG. 2C is a top plan view of the sensor of FIG. 2A having a plurality of sensing elements aligned in multiple directions;

FIG. 2D is a top plan view of the sensor of FIG. 2A having a first set of sensing elements aligned in a first direction and a second set of sensing elements aligned in a second direction;

FIG. 3 is a cross-sectional front view of the sensor of FIG. 2A;

FIG. 4A is a top plan view of the sensor of FIG. 2A having a plurality of sensing elements arranged in a first configuration;

FIG. 4B is a cross-sectional front view of the sensor of FIG. 3A;

FIG. 5A is a top plan view of the sensor of FIG. 2A having a plurality of sensing elements arranged in a second configuration;

FIG. 5B is a top plan view of the sensor of FIG. 2A having a plurality of sensing elements arranged in a third configuration;

FIG. 5C is a cross-sectional front view of the sensor of FIG. 5A;

FIG. 6A is a top plan view of the sensor of FIG. 5A having a plurality of sensing elements aligned in a first direction;

FIG. 6B is a top plan view of the sensor of FIG. 5A having a plurality of sensing elements aligned in a second direction;

FIG. 6C is a top plan view of the sensor of FIG. 5A having a plurality of sensing elements aligned in multiple directions;

FIG. 7A is a top plan view of the electrode of FIG. 1 having a stabilizing flap in accordance with an exemplary embodiment of the present disclosure;

FIG. 7B is a cross-sectional front view of the electrode of FIG. 7A;

FIG. 7C is a cross-sectional illustration of a spinal cord showing placement of the electrode of FIG. 7A;

FIG. 8A is a top perspective view of the electrode of FIG. 1 having a stabilizing flap and a plurality of stimulating elements in accordance with an exemplary embodiment of the present disclosure;

FIG. 8B is a cross-sectional illustration of a spinal cord showing placement of the electrode of FIG. 8A;

FIG. 9A is a top perspective view of the electrode of FIG. 1 coupled to another electrode by a stabilizing flap and each electrode having a plurality of stimulating elements in accordance with an exemplary embodiment of the present disclosure;

FIG. 9B is a cross-sectional illustration of a spinal cord showing placement of the electrode of FIG. 9A;

FIG. 10A is a top perspective view of an electrode having a stabilizing flap and a plurality of stimulating elements disposed on the stabilizing flap in a first configuration in accordance with an exemplary embodiment of the present disclosure;

FIG. 10B is a cross-sectional illustration of a spinal cord showing placement of the electrode of FIG. 10A;

FIG. 11A is a top perspective view of an electrode having a stabilizing flap and a plurality of stimulating elements disposed on the stabilizing flap in a second configuration in accordance with an exemplary embodiment of the present disclosure;

FIG. 11B is a cross-sectional illustration of a spinal cord showing placement of the electrode of FIG. 11A;

FIG. 12A is a top perspective view of an electrode having a stabilizing flap and a plurality of stimulating elements disposed on the stabilizing flap in a third configuration in accordance with an exemplary embodiment of the present disclosure;

FIG. 12B is a cross-sectional illustration of a spinal cord showing placement of the electrode of FIG. 12A;

FIG. 13A is a top perspective view of an electrode having a stabilizing flap and a plurality of stimulating elements disposed on the stabilizing flap in a fourth configuration in accordance with an exemplary embodiment of the present disclosure;

FIG. 13B is a cross-sectional illustration of a spinal cord showing placement of the electrode of FIG. 13A;

FIG. 14A is a top perspective view of an electrode having a plurality of stimulating elements in accordance with an exemplary embodiment of the present disclosure; and

FIG. 14B is a cross-sectional illustration of a spinal cord showing placement of the electrode of FIG. 14A.

FIG. 15 depicts the physical setup of the sensor.

FIG. 16 is a flow chart of the communication flow.

FIG. 17 is a simplified side cutaway view of the sensors as they would be inserted.

FIG. 18 depicts a dorsal view of the test set-up.

FIG. 19 depicts a lateral view of the test set-up.

FIG. 20 depicts a lateral view of the test set-up, with the model in a flexed position.

FIG. 21 depicts a lateral view of the model with the model in an extension position.

FIG. 22 depicts the flex sensor values during the testing procedure, and the threshold used to determine the position.

FIG. 23 depicts a 2.5 second time window of the stimulation pulse during one transition between extension and flexion.

FIGS. 24A-24D depict the flex sensors in various states movement.

FIG. 25 depicts the flex sensor values.

FIG. 26 shows a 6 second time window of the stimulation pulse during one transition between rest and both directions of torsion.

FIG. 27 depicts the data collected from the continuous stimulation modulation testing procedure.

FIG. 28 depicts a zoomed in 6 second window of the stimulation pulse during a change between flexion and extension.

FIG. 29 depicts flex sensor values during our testing procedure and highlighted regions to indicate the state.

FIG. 30 depicts a sample stimulation pulse during the two torsion states.

FIG. 31 depicts a diagram of an Inertial Movement Unit (IMU).

FIG. 32 is a flow chart of the communication in the system

FIG. 33 depicts the IMU in place on the head of a test subject.

FIG. 34 depicts a motion guide for use in testing the IMU.

FIGS. 35A-35I depict the various head movements

FIG. 36 depicts the IMU quaternion data.

FIG. 37 depicts the stimulation pulse throughout a 30 second window of the experiment.

FIG. 38 depicts a zoomed in section of FIG. 37 to shown that the dense black regions of that plot are high frequency stimulation pulses.

FIG. 39 depicts the IMU quaternion data as it changes throughout the trail.

FIG. 40 shows the stimulation pulse throughout a 30 second window of the experiment.

FIG. 41 depicts a zoomed in section from FIG. 40 to shown that the dense black regions of that plot are high frequency stimulation pulses.

### DETAILED DESCRIPTION

Spinal cord stimulation (“SCS”) devices may be used to provide stimulation to the spinal cord for treatment of neurological diseases and disorders such as chronic pain and/or stroke, Spinal Cord Injury (SCI), Spinal Muscular Atrophy (SMA), Traumatic Brain Injury (TBI), Cerebral Palsy (CP), Amyotrophic Lateral Sclerosis (ALS), and/or Duchenne Motor Disorder. SCS devices may provide stimulation to the spinal cord to treat impairment of a limb due to the neurological disease or disorder. However, when providing stimulation to the spinal cord, current SCS devices may not account for movement of the spine relative to the SCS device. Movement of the spine may result in inadequate or improper stimulation or stimulation to the wrong area and current SCS devices are unable to detect when the spine moves and thus are unable to adjust their stimulation parameters accordingly. The functional effect of the stimulation provided by the SCS device may be altered due to movement of the SCS device relative to the spinal cord. Further, current SCS devices do not secure the SCS device in place and/or prevent movement of the SCS device relative to the spinal cord when providing specific stimulation to a targeted area. Movement of the SCS device relative to the spinal cord can affect the therapeutic effect of the stimulation provided to the spinal cord.

A method of detecting movement of a spine, and stimulating specific regions of the spinal cord. The method includes providing a sensor having one or more sensing elements to sense movement of the spine. Providing one or more stimulating elements configured to electrically stimulate an area proximate the spine. Detecting, via the sensor, a movement of the spine. Modulating an electrical stimulus provided by the one or more stimulating elements at an area proximate the spine in response to the detected movement.

A sensor and stimulator. The sensor and stimulator comprising a sensor body and a stimulator body. The stimulator body having a first end and a second end opposite the first end, the stimulator body configured to be placed proximate to a spine of a patient. A plurality of sensing elements disposed on the sensor body. One or more stimulating elements configured to electrically stimulate an area proximate the spine. A control circuit disposed proximate the second end of the stimulator body, wherein the control circuit is configured to 1) measure an initial property associated with the sensor being in an initial state; 2) detect change from the initial property; 3) measure, upon detecting the change, an altered property associated with the sensor being in an altered state; 4) correlate the altered property associated with the sensor to movement of the spine of the patient; and 5) modulate the electrical stimulus provided by the one or more stimulating elements at an area proximate the spine responsive to the altered property.

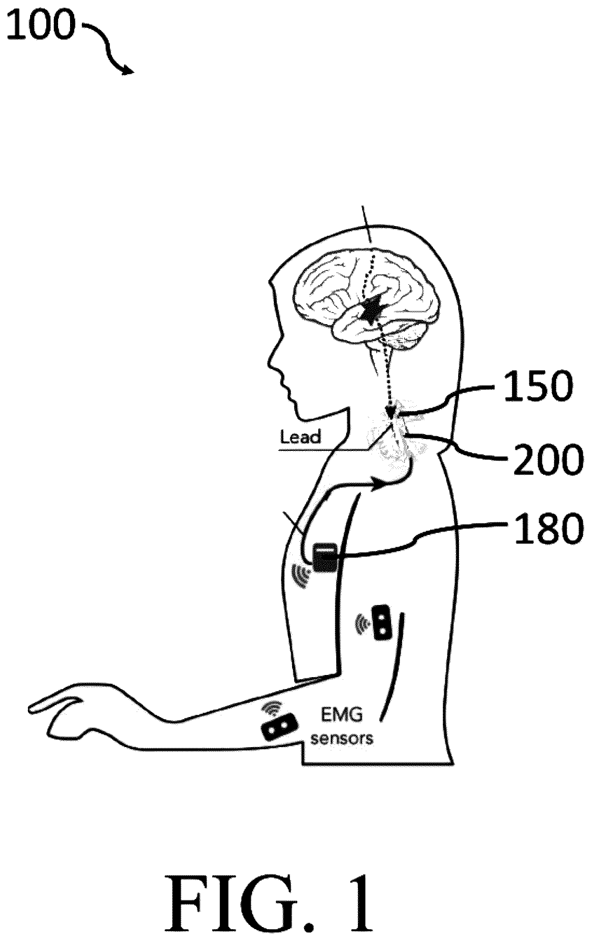

Exemplary embodiments of the present disclosure provide a spinal cord stimulation device, generally designated as device 100. Embodiments of the present disclosure are shown as exemplary device 100 having sensor 150 and/or electrode 200, as shown in FIGS. 1-14B. In use, device 100 provides stimulation to the spinal cord of a patient or an area proximate the spinal cord. Device 100 may be configured to treat impairment of a limb of the patient due to a neurological disease or disorder. For example, a patient may have a limb that has a motor impairment and/or proprioception impairment due to a stroke or another type of neurological disorder or disease. Device 100 may be configured to provide stimulation to an area proximate the spinal cord, such as the dorsal root, to reduce the impairment of the affected limb. In some embodiments, neurons that comprise the dorsal root innervate the affected limb and interact with other neurons that affect movement in the affected limb; stimulation of the dorsal root provides a therapeutic effect to the affected limb.

Referring to FIG. 1, device 100 may include neurostimulator or control circuit 180, sensor 150, and/or electrode 200. In some embodiments, sensor 150 and electrode 200 are referred to as leads or spinal leads. In some embodiments, neurostimulator 180 is coupled to one or more of sensor 150 and electrode 200. Neurostimulator 180 may be configured to provide a stimulation to the spinal cord or an area proximate the spinal cord via electrode 200. In some embodiments, each of neurostimulator 180, sensor 150, and electrode 200 are implanted within the patient. Sensor 150 and/or electrode 200 may be implanted within the patient such that sensor 150 and/or electrode 200 are disposed proximate the spinal cord of the patient, such as within the epidural space. In some embodiments, sensor 150 and/or electrode 200 are implanted within epidural space of the patient proximate the dorsal root. Sensor 150 and/or electrode 200 may include radiopaque markers or other types of markers or indicators to assist with placement and implantation of sensor 150 and/or electrode 200 within the body of the patient. Sensor 150 may include a plurality of sensing elements configured to detect electrical properties of sensor 150 to determine flexing and/or twisting of sensor 150. Electrode 200 may include a plurality of stimulating elements configured to provide electrical stimulation to a desired target area or record electrical activity from a desired target area. The desired target area may be proximate electrode 200.

In some embodiments, sensor 150 and/or electrode 200 are disposed proximate the dorsal root of the spinal cord. Sensor 150 may include electrode 200 or electrode 200 may include sensor 150. For example, sensor 150 may include both sensing elements configured to detect movement (e.g., flexing and twisting) of the spine and stimulating elements to provide electrical stimulation to or record electrical signals from a desired area. In some embodiments, the stimulating elements are also recording elements configured to record electrical activity or electrical signals from a desired area. Alternatively, electrode 200 may include both stimulating elements to provide electrical stimulation to or record electrical signals from a desired area and sensing elements configured to detect movement (e.g., flexing and twisting) of the spine. Movement of the spine may be due to flexing or contracting of one or more muscles and/or skeletal structures located proximate the spine of the patient. This can be due to voluntary motor control or involuntary motor control.

Neurostimulator 180 may be configured to receive sensory data from sensor 150. The sensory data from sensor 150 may include electrical property values that can be correlated to movement (e.g., flexing, twisting, torsion) of the spine or neck. Neurostimulator 180 may include a processor or control circuitry configured to transmit controls to sensor 150 and/or electrode 200 and receive data from sensor 150 and/or electrode 200. In some embodiments, neurostimulator is configured to provide electrical signals to electrode 200 to provide electrical stimulation to a desired area. In some embodiments, sensor 150 and/or electrode 200 includes a control circuit disposed within. For example, sensor 150 and/or electrode 200 may include a control circuit disposed upon sensor 150 and/or electrode 200.

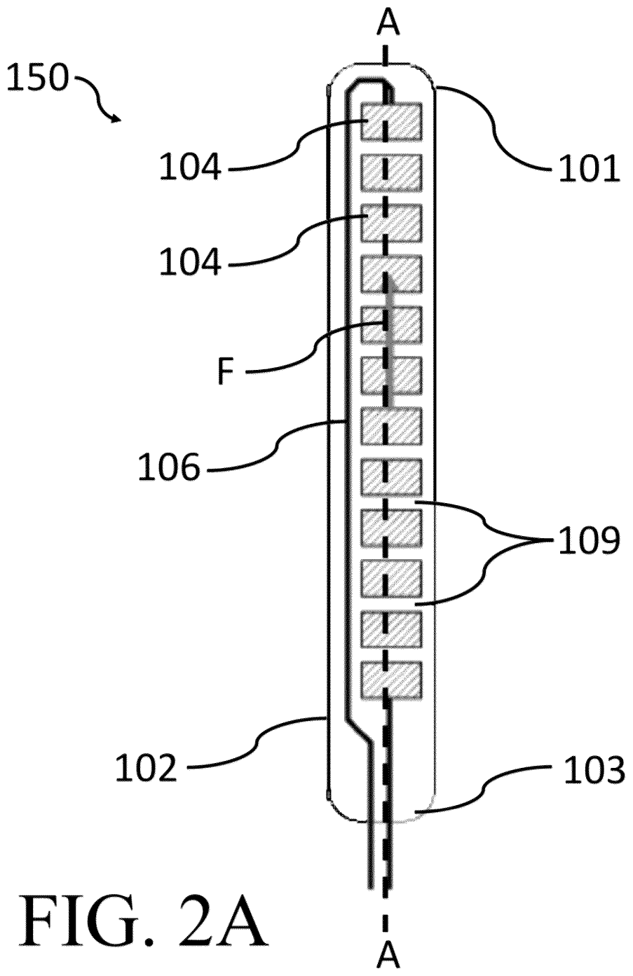

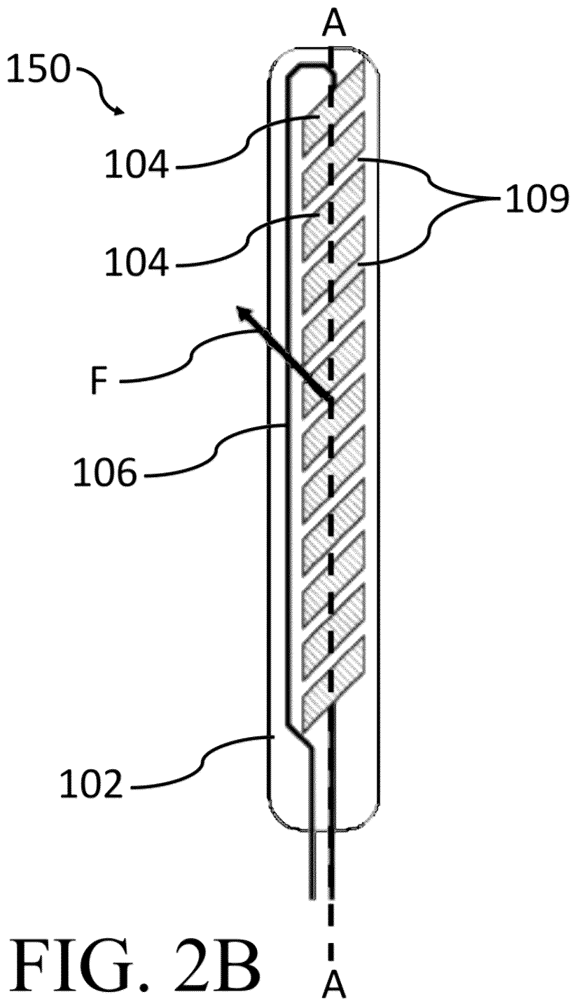

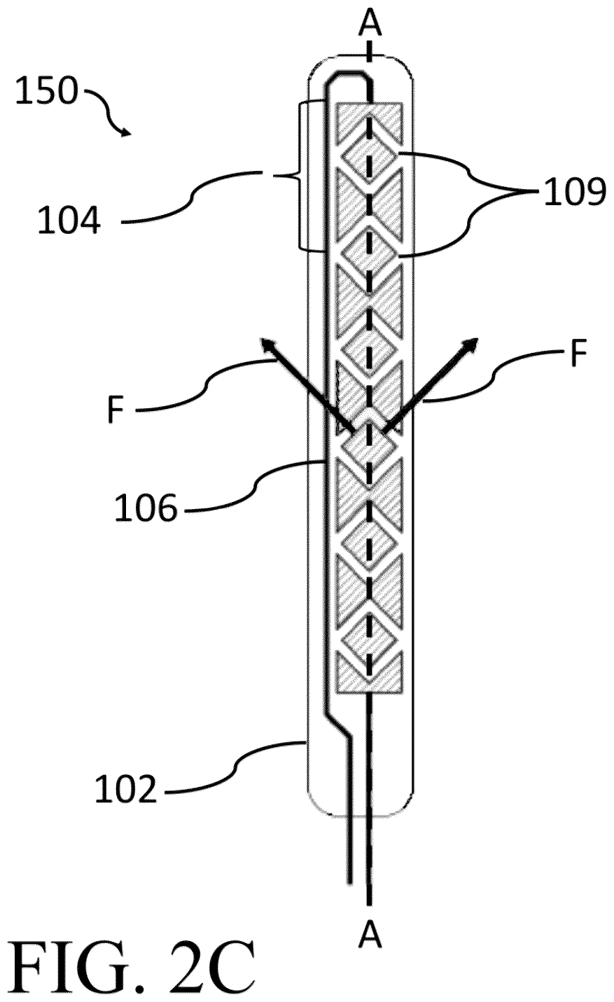

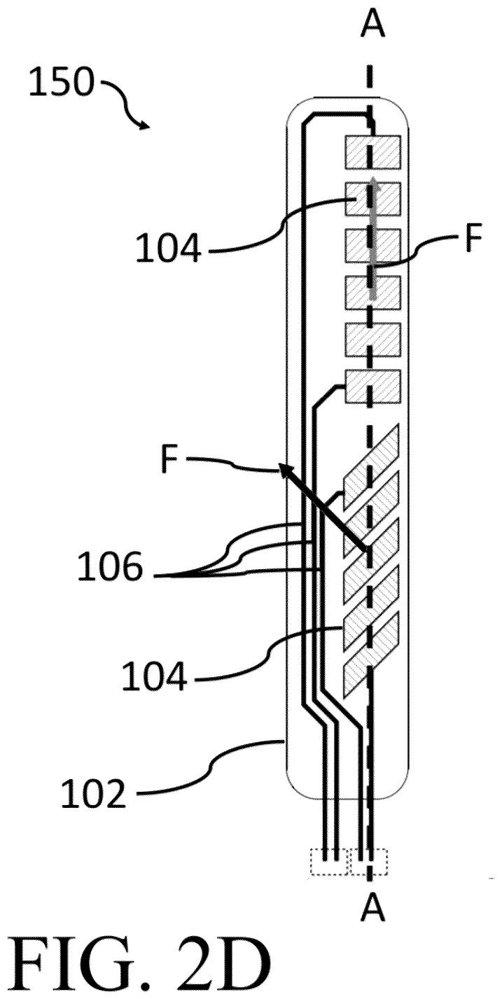

Referring to FIGS. 2A-6C, device 100 may include sensor 150. Sensor 150 may be implanted within the patient proximate the spinal cord and may be configured to detect movement of the spine. For example, sensor 150 may be implanted within the patient such that sensor 150 is disposed proximate the spinal cord (e.g., the dorsal roots). In some embodiments, sensor 150 is implanted into the patient via, for example, a laminectomy to expose the epidural space and allow for placement of sensor 150 within the epidural space. Sensor 150 may be configured to alter its electrical properties upon deformation, which may be correlated to movement (e.g., flexing, twisting, torsion, flexion, extension, rotation, lateral bending) of the head and neck.

Sensor 150 may include body 102, a plurality of sensing elements 104, and conductive wire 106. Body 102 may include first end 101 and second end 103, and longitudinal axis A-A which extends through first end 101 and second end 103. First end 101 may be opposite second end 103 and longitudinal axis A-A may extend through first end 101 and second end 103. In some embodiments, body 102 has a length disposed between first end 101 and second end 103, and a width that is transverse to longitudinal axis A-A. In some embodiments, conductive wire or wire 106 couples one or more sensing elements 104 together. Sensor 150 may include or be coupled to a control circuit (e.g., neuromodulator or control circuit 180), which may be configured to measure deformation based on an electrical property of sensor 150. The control circuit may be configured to sense deformation of sensor 150 based on change in the resistance value of sensor 150

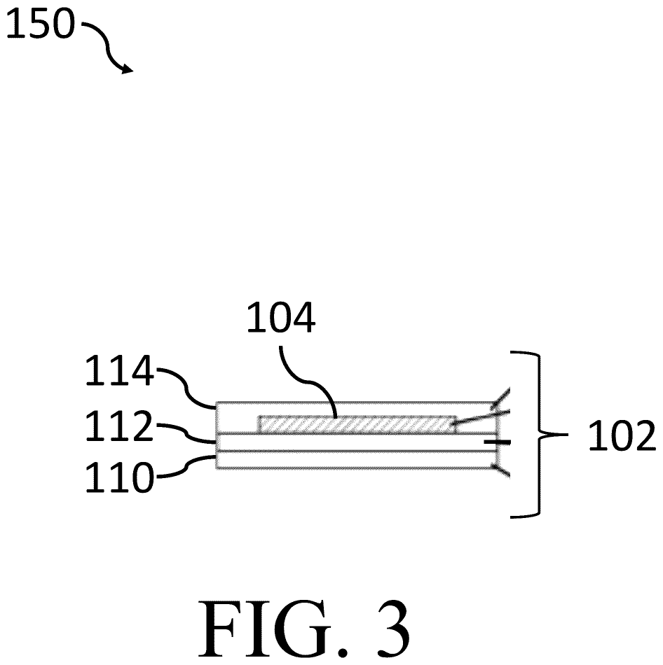

Referring to FIGS. 2A-3, body 102 of sensor 150 may include substrate layer 110, resistive layer 112, and overmolding 114. Resistive layer 112 may be disposed between substrate layer 110 and overmolding 114. In some embodiments, sensing elements 104 are disposed on top of resistive layer 112 and within overmolding 114. Resistive layer 112 may be configured to change resistance upon deformation. For example, deformation of sensor 150 may result in deformation of resistive layer 112, which may change the resistance of resistive layer 112 and thereby change the resistance of sensor 150. The resistance value may be measured across sensor 150 by a control circuit (e.g., neuromodulator or control circuit 180). In some embodiments, sensing elements 104 are arranged on body 102 in series with one or more gaps 109 between each sensing element 104. The resistance value measured for sensor 150 may be dependent on resistive layer 112. In some embodiments, gaps 109 between each sensing element 104 is varied to change the sensitivity of the resistive changes of sensor 150. The greater the combined length of the gaps, the larger the undeformed resistance value will be. If the gaps are too big or too small the ratio of the difference in resistance and the undeformed initial resistance and/or the sensitivity to deformations, will be non-optimal.

In some embodiments, resistive layer 112 is formed by using conductive ink deposited on substrate layer 110. Substrate layer 110 may be comprised of a flexible substrate. In some embodiments, substrate layer 110 is a polymeric substrate. Resistive layer 112 may be formed using conductive ink or particles embedded within substrate layer 110, which may be a silicone material.

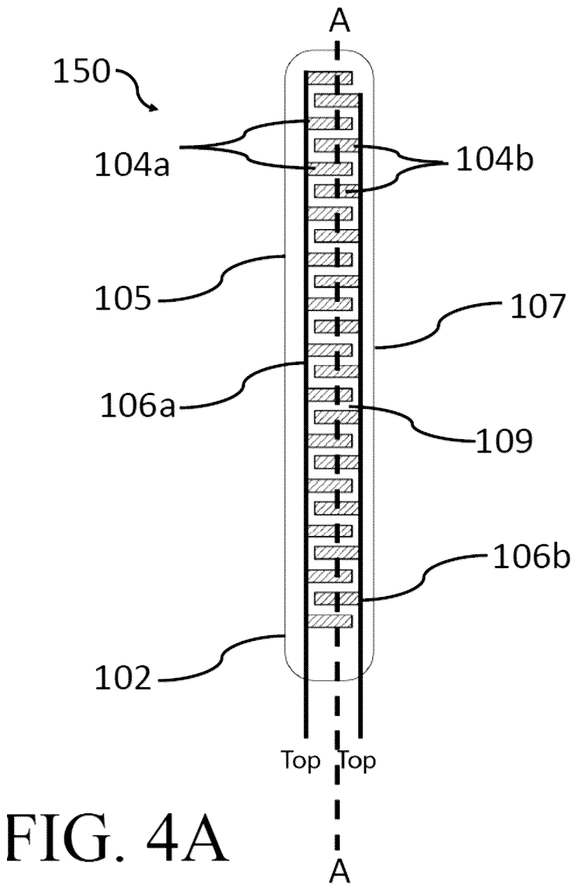

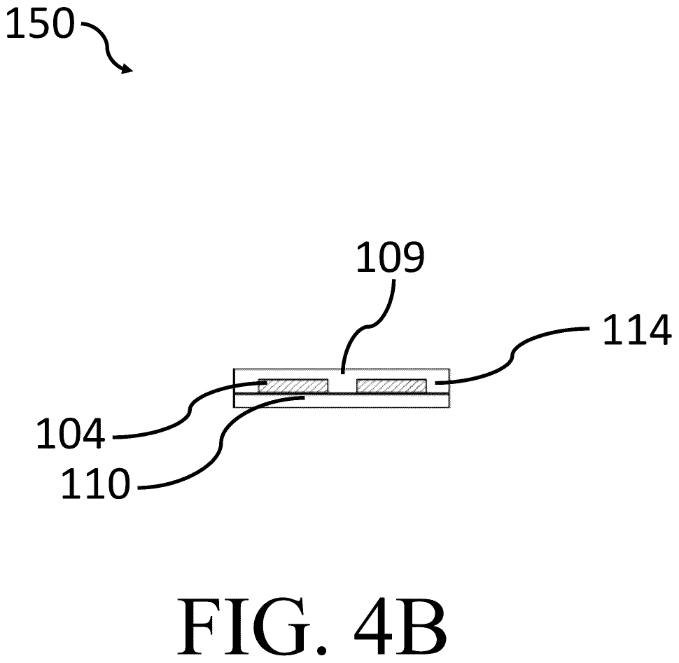

Referring to FIGS. 2A-6C, the plurality of sensing elements 104 may be independent or coupled together via conductive wire 106. In some embodiments, sensing elements 104 are coupled together via wire 106, which is conductive, such that each sensing elements 104 is electrically coupled to an adjacent sensing element 104 to form a larger unitary sensing element, such as sensing element 104a and 104b (as shown in FIG. 4A). For example, wire 106 may couple one or more sensing elements 104 together such that one or more sensing elements 104 form a circuit within sensor 150. Sensor 150 may include one or more gaps 109 disposed between each sensing element 104. Gaps 109 may comprise insulating material, conductive material, or partially conductive material. Sensor 150 may have an electrical property value (e.g., resistance value, capacitance value, voltage value, current value) due to sensor 150 being comprised of one or more sensing elements 104 spaced by gaps 109 and sensing elements 104 being coupled together via wire 106. The electrical property of sensor 150 may change due to deformation of sensor 150. The electrical property value may be a resistance value, or a capacitance value associated with sensor 150.

Sensing elements 104 may be conductive elements comprised of one or more of metal, conductive epoxy, or any other conductive material. Sensing elements 104 may be disposed along body 102 and may be disposed between first end 101 and second end 103. For example, sensor 150 may include one sensing element 104 proximate first end 101 and another sensing element 104 proximate second end 103, with wire 106 coupling each sensing element 104. In this embodiment, the resistance of sensor 150 is the resistance of restive layer 112 between sensing element 104 proximate first end 101 and sensing element 104 proximate second end 103. In some embodiments, additional sensing elements 104 (with gaps 109 disposed between adjacent sensing elements 104) are used to increase the sensitivity of sensor 150.

In some embodiments, sensor 150 includes one or more sensing elements 104 disposed between first end 101 and second end 103. Sensing elements 104 may be spaced apart on sensor 150 but coupled together via wire 106 to maximize the surface area of sensor 150 that sensing elements 104 cover on sensor 150. In practice, gaps 109 between sensing elements 104 allow for movement and deformation of resistive layer 112, which varies the resistance of sensor 150. Each gap 109, which exposes resistive layer 112, may be analogous to a variable resistor since the resistance of resistive layer 112 between each sensing element 104 varies when sensor 150 is deformed. The total resistance of sensor 150 may be determined based on the combination of resistance values at each exposed portion of resistive layer 112 (e.g., each gap 109). As the total resistance value of sensor 150 varies, control circuit is configured to calculate the deformation of sensor 150.

In some embodiments, sensor 150 has an initial state and an altered state. The initial state and altered state may be associated with deformation of sensor 150. For example, the initial state is when sensor 150 is undeformed and the altered state is when sensor 150 is deformed. The initial state may be a resting or baseline state (e.g., position) of sensor 150. The altered state may be when sensor 150 has deviated in position or shape from the initial state, which can result from deformation of sensor 150. For example, deformation of sensor 150 may result in sensor 150 changing shape (e.g., twisting or flexing) causing sensor 150 to move from the initial state to the altered state. In some embodiments, flexing or twisting of the spine causes deformation of sensor 150, which results in sensor 150 moving from the initial state to the altered state. In the initial state, the control circuit (e.g., neuromodulator or control circuit 180) may measure an initial electrical property value. Upon deviation of sensor 150 to the altered state, the control circuit (e.g., neuromodulator or control circuit 180) which is functionally connected to the sensor 150 may measure an altered electrical property value associated with sensor 150 being in the altered state (e.g., deformed).

In some embodiments, the altered electrical property value may be correlated with movement (e.g., flexing or twisting) of the spine. For example, the altered electrical property value (e.g., resistance value or capacitance value) may be compared to a threshold value to determine when sensor 150 has moved from the initial state to the altered state upon deformation. The threshold value may be an electrical property value of sensor 150 when sensor 150 is in the initial state or an electrical property value of sensor 150 that is substantially close in value to the initial electrical property value of when sensor 150 is in the initial state. Having the threshold value be close to the initial electrical property value of when sensor 150 is in the initial state reduces false positives and provides a buffer between the initial electrical property value and the altered electrical property value. When it is determined that that the altered electrical property value exceeds the threshold value, the difference between the altered electrical property value and the threshold value may be calculated. Based on the difference, a spinal deviation is calculated. In some embodiments, the spinal deviation includes one or more of flexing or twisting of the spine from an initial spinal position to a deviated spinal position.

In some embodiments, correlating the altered electrical property value with the movement of the spine includes continuously or periodically measuring the altered electrical property value of sensor 150. The difference between the altered electrical property value and the initial electrical property value may be calculated, such as by the control circuit (e.g., neuromodulator or control circuit 180). Based on the difference, a spinal deviation may be continuously or periodically calculated. For example, the greater the difference the greater the deformation of sensor 150 and thus the greater the flexing and/or twisting of the spine.

Referring to FIGS. 2A-2D, sensing elements 104 may be arranged on body 102 of sensor 150 to measure flexing and twisting of the spine. For example, sensing elements 104 may be placed horizontally (e.g., spanning the width of sensor 150) to measure flexion. To measure twisting of the spine, sensing elements 104 may be arranged such that the gaps between each sensing element 104 and sensing elements 104 themselves are aligned with the direction of the largest deformation, denoted as “F”. For example, sensing elements 104 may be arranged diagonally (e.g., FIG. 2B) relative longitudinal axis A-A when deformation force F is at an angle relative to longitudinal axis A-A. Arranging sensing elements 104 diagonally relative to longitudinal axis A-A may result in sensor 150 being able to simultaneously detect changes in resistance values caused by flexion and twisting. In some embodiments, in addition to the amount of deformation, the direction of deformation of sensor 150 is also measured. For example, as sensor 150 deforms gaps 109 may increase or decrease in size resulting in resistive layer 112 also deforming and changing the resistance value of sensor 150. In other words, resistive layer 112 of sensor 150 is anisotropic resulting in sensor 150 being able to determine the direction of deformation due to the electrical property value measured (e.g., resistance value).

In use, to determine movement of the spine, such as flexing and/or twisting, the deformation of sensor 150 must be determined. To quantify the amount of deformation of sensor 150, the resistance value of sensor 150 is measured. In some embodiments, sensor 150 is placed in series with a fixed resistor that has a similar resistance value as sensor 150. Sensor 150 and the fixed resistor having a similar resistance as sensor 150 may be arrange in a voltage divider configuration. In some embodiments, the voltage is measured at a node between sensor 150 and the fixed resistor. The measured voltage may be proportional to the resistance value of sensor 150 and thus may provide an indication of deformation of sensor 150. For example, the voltage may be measured between sensor 150 and the fixed resistor when sensor 150 is in the initial state. Upon deformation of sensor 150 and sensor 150 being in the altered state, the voltage measured between sensor 150 and the fixed resistor changes indicating deformation of sensor 150.

In some embodiments, the resistance value of sensor 150 is measured using a Wheatstone bridge circuit formed by sensing elements 104 and gaps 109. Sensor 150 may be included in a differential circuit with 3 other fixed resistors. Sensor 150 in the Wheatstone bridge circuit may provide greater sensitivity to deformation than when sensor 150 is in series with a fixed resistor. In some embodiments, sensor 150 in series with a fixed resistor and sensor 150 in a Wheatstone bridge circuit each require an external voltage that is applied to sensor 150 and the other fixed resistors, passive electrical components such as other resistors, and the ability to measure an output voltage.

In some embodiments, sensor 150 includes a control circuit. For example, sensor 150 may include a control circuit coupled to sensor 150 or disposed on sensor 150 proximate second end 103 of body 102. The control circuit may be configured to measure electrical property values (e.g., voltage values, resistance values, capacitance values, impedance values) of sensor 150 and/or the fixed resistors. In some embodiments, neurostimulator 180 includes the control circuit and is communication with sensor 150.

In some embodiments, sensing elements 104 and gaps 109 are configured to have a primary direction (deformation force F). Due to the primary direction F, the difference between the initial electrical property of sensor 150 and the altered electrical property of sensor 150 may be different for some deformations of sensor 150 than it is for other deformations of sensor 150. Sensor 150 may contain one or more sets of sensing elements 104 and gaps 109 each set with different primary directions F (e.g., different shapes) and which have different electrical property values for the same altered state of the sensor 150. For example, a first set of sensing elements 104 and gaps 109 may be sensitive to flexing and a second set of sensing elements 104 and gaps 109 may be sensitive to twisting. Further, because both are present on sensor 150, both deformations can be identified separately.

Sensor 150 may include a first set of sensing elements 104 coupled together via first wire 106, each spaced apart by a first set of gaps 109 and sensor 150 may also include a second set of sensing elements 104 coupled together via a second wire 106, each spaced apart by a second set of gaps 109. The first set of sensing elements 104, gaps 109, and first wire 106 may comprise a first circuit and the second set of sensing elements 104, gaps 109, and second wire 106 may comprise a second circuit. The first circuit may be configured to detect deformation along a first direction and the second circuit may be configured to detect deformation long a second direction, which may be different than the first direction. A single sensor 150 may include both the first circuit and the second circuit to deformation along multiple directions. In some embodiments, electrical property values from the first circuit and the second circuit are combined to generate an electrical property value of sensor 150. In some embodiments, the first circuit and the second circuit are each independently coupled to a control circuit (e.g., neuromodulator or control circuit 180).

In some embodiments, the shape of sensing elements 104 varies based on the deformation sensitivity desired. For example, sensing elements 104 may be substantially symmetrical and may be arranged on sensor 150 such that longitudinal axis A-A bifurcates one or more sensing elements 104 and one or more sensing elements 104 are symmetrical about longitudinal axis A-A. In some embodiments, sensing elements 104 are substantially symmetrical about longitudinal axis A-A (e.g., rectangular, square, oval, circular, triangular, diamond, hexagonal, octagonal, arrow shaped, bowtie) and arranged on sensor 150 such that each sensing elements 104 is symmetrical about longitudinal axis A-A. Sensing elements 104 being substantially symmetrical about longitudinal axis A-A, such as shown in FIG. 2A, results in sensor 150 being sensitive to deformation force F when it aligns with or is substantially parallel to longitudinal axis A-A. Deformation force F may be the major force felt by sensor 150 resulting in deformation of sensor 150. In some embodiments, sensing elements 104 being substantially symmetrical about longitudinal axis A-A allows sensing elements 104 to be sensitive to deformation force F when it is substantially parallel to longitudinal axis A-A and when deformation force F is at an angle relative to longitudinal axis A-A (e.g., FIG. 2C).

In some embodiments, sensing elements 104 are non-symmetrical or angled to a lateral side of body 102, such as shown in FIG. 2B. Sensing element 104 being angled to a lateral side of body 102 (e.g., FIG. 2B) results in sensor 150 being sensitive to deformation force F when at an angle to longitudinal axis A-A. In some embodiments, sensing elements 104 are arranged in multiple shapes throughout sensor 150 such that sensing elements 104 are sensitive when deformation force F is in multiple directions, such as in FIGS. 2C and 2D. In some embodiments, sensor 150 is configured to detect deformation forces F due to both twisting and bending. Sensing elements 104 may be substantially symmetrical about longitudinal axis A-A to allow sensing elements 104 to be sensitive to deformation force F when deformation force F is at various angles relative to longitudinal axis A-A. For example, sensing elements 104 may be a combination of one or more shapes symmetrical about longitudinal axis A-A, as shown in FIG. 2C, such that sensing elements 104 is sensitive to when deformation angle Fis at various angles relative to longitudinal axis A-A. In some embodiments, sensing elements 104 are arranged in any combination of shape and/or configuration discussed herein and multiple sensors 150 with various arrangements of sensing elements 104, such as those discussed herein, are implanted into the patient to detect various movements of the spine.

Referring to FIGS. 4A-6C, the electrical property value measured by the control circuit (e.g., neuromodulator or control circuit 180 may be a capacitance value of sensor 150 and may vary based on the deformation of sensor 150. The capacitance of sensor 150 may change upon sensor 150 moving from the initial state to the altered state (e.g., upon deformation of sensor 150 caused by flexing/twisting of the spine). In this configuration, body 102 may not include resistive layer 112. For example, body 102 of sensor 150 may include substrate layer 110 and overmolding 114, and sensing elements 104 may be disposed on top of substrate layer 110 and within overmolding 114 (e.g., FIG. 4B). The capacitance of sensor 150 may be determined based on the distance between sensing elements 104. Each sensing element 104 may include a metal plate, and each sensing element 104 with an adjacent sensing elements 104 may comprise a capacitor with overmolding 114 and/or substrate layer 110 serving as a dielectric material between sensing elements 104 (e.g., metal plates). In use, as sensor 150 moves from the initial state to the altered state due to deformation of sensor 150, the distance between one sensing element 104 and an adjacent sensing element 104 changes, thereby changing the capacitance value of sensor 150. In some embodiments, sensor 150 includes one or more gaps 109 disposed between sensing elements 104. For example, sensor 150 may include gap 109 disposed between adjacent sensing elements 104. In some embodiments, sensing elements 104 includes first set of sensing elements (“first set”) 104a and second set of sensing elements (“second set”) 104b. First set 104a and second set 104b may both be arranged on a top surface of sensor 150, such as shown in FIGS. 4A-4B. First set 104a may be arranged on body 102 such that it extends from an area proximate first side 105 and second set 104b may be arrange on body 102 such that it extends from an area proximate second side 107. In some embodiments, first set 104a may alternate with second set 104b such that a sensing element from second set 104b is disposed between two adjacent sensing elements of first set 104a (e.g., FIG. 4A). In this configuration, sensor 150 may include gap 109 on the top surface. Gap 109 may be continuous due to first set 104a and second set 104b alternating. Deformation of sensor 150 may cause first set 104a to move relative to second set 104b changing the distance (e.g., gap 109) between the sensing elements of first set 104a and the sensing elements of second set 104b. First set 104a moving relative to second set 104b may result in a change in capacitance of sensor 150. For example, as sensor 150 moves from the initial state to the altered state due to deformation of sensor 150, first set 104a and second set 104b may move relative to each other resulting in a change in the capacitance value of sensing elements 104 and sensor 150.

In some embodiments, sensing elements 104 of first set 104a are coupled together via first wire 106a and sensing elements 104 of second set 104b are coupled together via second wire 106b. First set 104a may form a first plate of a capacitor and second set 104b may form a second plate of the capacitor such that the capacitance of sensor 150 is based on the capacitance value of the capacitor formed by first set 104a and second set 104b.

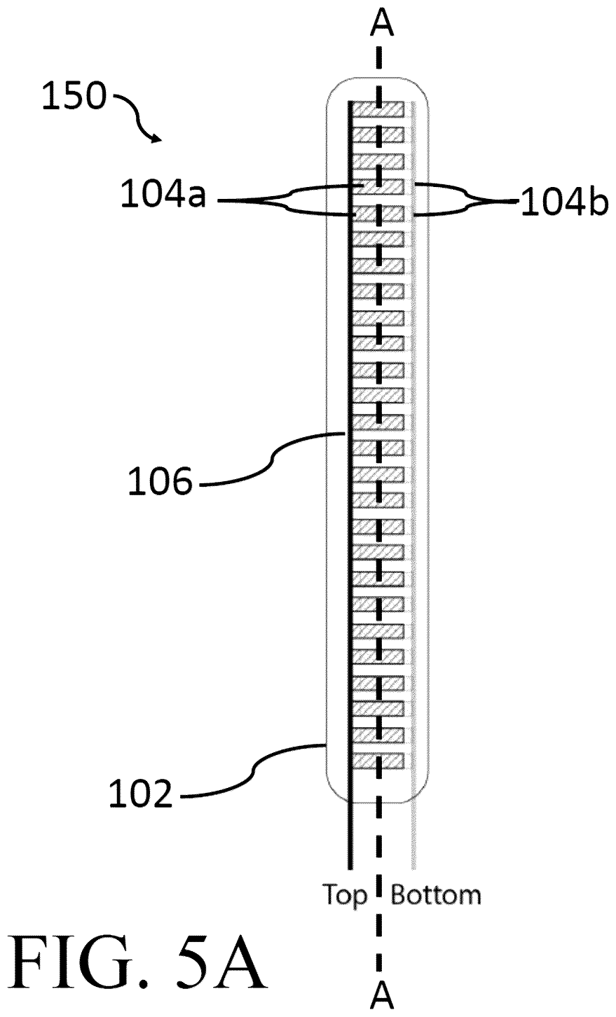

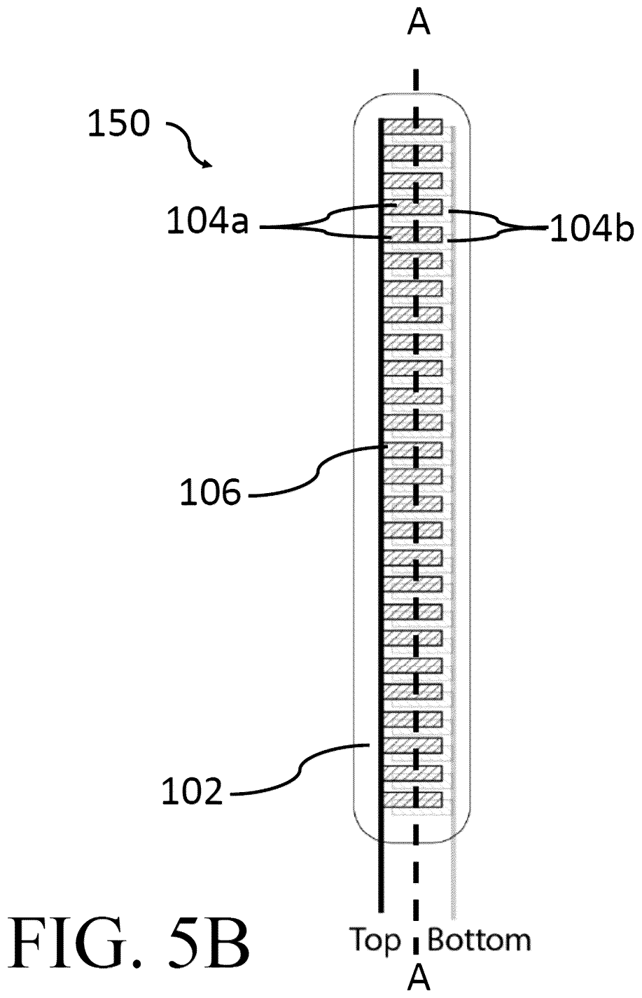

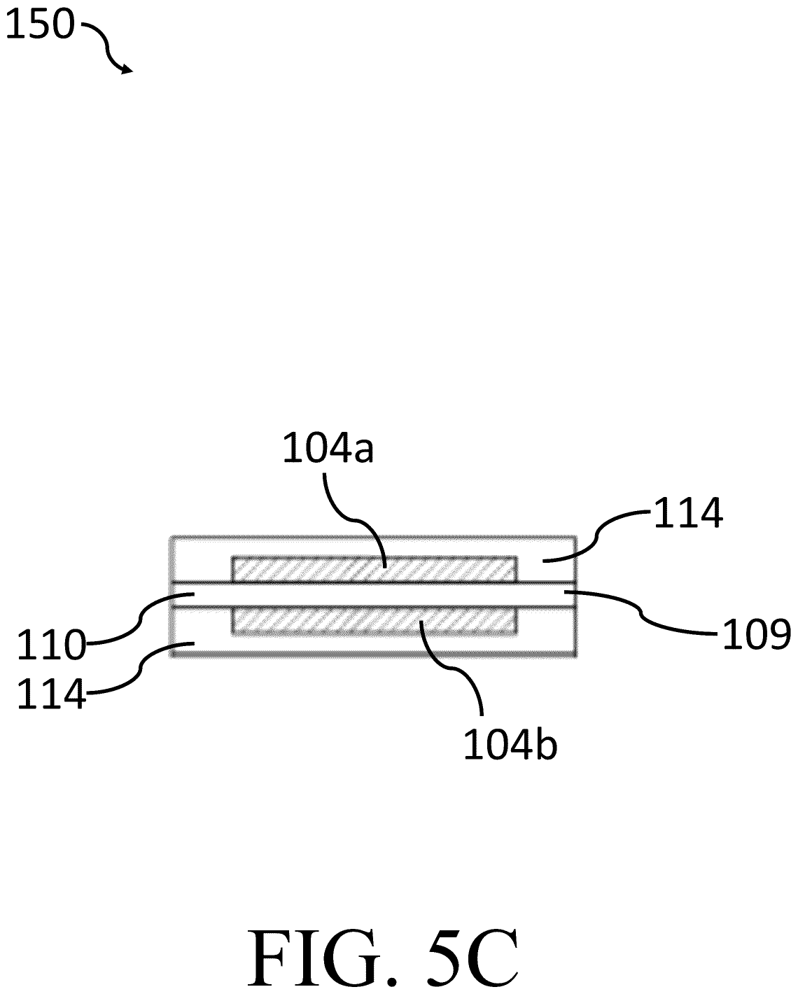

In some embodiments, first set 104a is disposed on a top surface of sensor 150 and second set 104b is disposed on a bottom surface of sensor 150 (e.g., FIGS. 5A-5C). In this configuration, body 102 may include an insulating layer, which may be substrate layer 110, overmolding 114, or another layer of body 102. Sensor 150 may include gap 109 disposed between the top surface (first set 104a) and bottom surface (second set 104a). Gap 109 may be varied to change the sensitivity of sensor 150. For example, as shown in FIG. 5C, gap 109 may be disposed between first set 104a and second set 104b. When sensor 150 is in the initial state, first set 104a may be aligned with second set 104b, as shown in FIG. 5A. Upon deformation of sensor 150 due to movement of the spine (e.g., twisting and/or flexing), sensor 150 may move from the initial state to the altered state causing one or more sensing elements of first set 104a to be misaligned or offset with one or more sensing elements of second set 104b, as shown in FIG. 5B. Upon first set 104a being misaligned or offset with second set 104b, the capacitance value of sensor 150 changes (e.g., decreases) indicating deformation of sensor 150 and movement (e.g., flexing and/or twisting) of the spine. In practice, having a plurality of sensing elements 104 instead of a single sensing element 104 allows for sensor 150 to be more sensitive to changes in capacitance when the plurality of sensing elements 104 are misaligned or offset. In other words, more sensing elements 104 allows for more overlapping surface area of sensor 150, which allows sensor 150 to be more sensitive to changes in electrical properties, such as capacitance and resistance.

In some embodiments, the capacitance value of sensor 150 is minimized when sensor 150 is in the initial state (e.g., unbent and/or undeformed) and is maximized when sensor 150 is in the altered state (e.g., deformed due to flexing and/or twisting of the spine). In some embodiments, the greater the difference in the capacitance value between sensor 150 being in the initial state and sensor 150 being in the altered sate, the greater the sensitivity of sensor 150 to deformation.

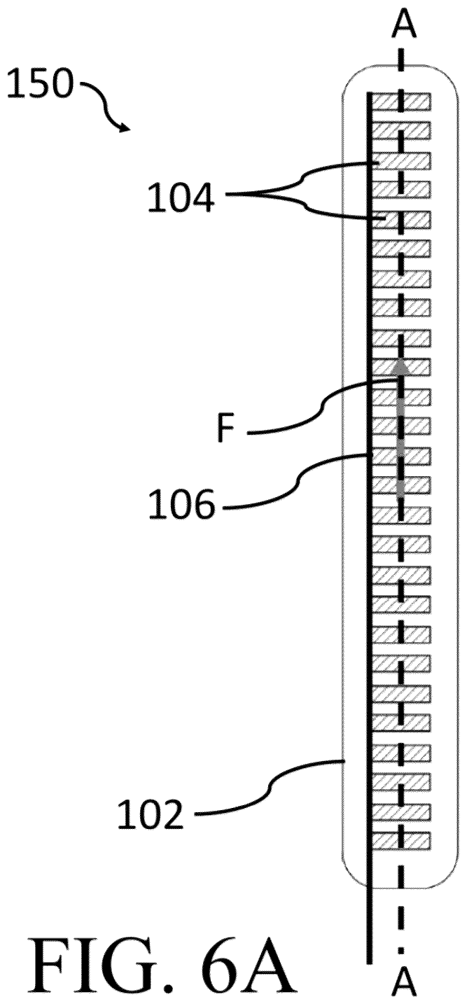

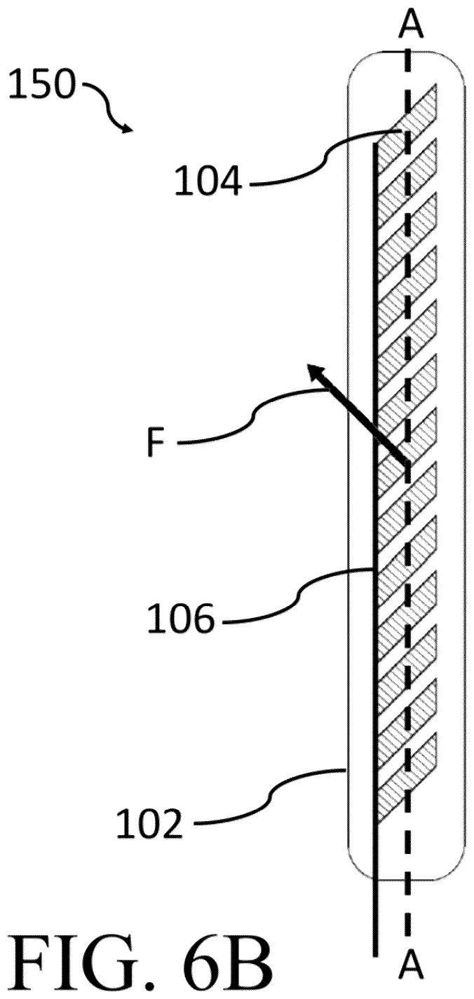

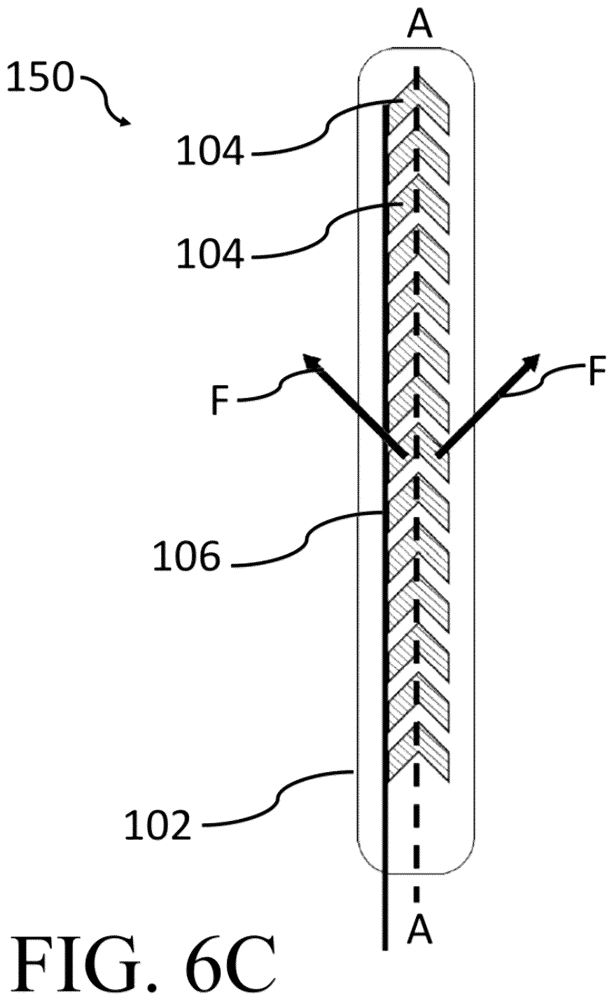

Referring to FIGS. 6A-6C, the configuration and alignment of sensing elements 104, including first set 104a and second set 104b, may be arranged based on deformation force F. In some embodiments, the shape of sensing elements 104 varies based on deformation sensitivity desired. For example, sensing elements 104 of first set 104a and/or second set 104b may be substantially symmetrical and may be arranged on sensor 150 such that longitudinal axis A-A bifurcates sensing elements 104 and sensing elements 104 are symmetrical about longitudinal axis A-A. In some embodiments, sensing elements 104 are substantially symmetrical (e.g., rectangular, square, oval, circular, triangular, diamond, hexagonal, octagonal, arrowhead shaped, bowtie) and arranged on sensor 150 such that each sensing elements 104 is symmetrical about longitudinal axis A-A. Sensing elements 104 being substantially symmetrical about longitudinal axis A-A, such as shown in FIG. 6A, results in sensor 150 being sensitive to deformation force F when it aligns with or is substantially parallel to longitudinal axis A-A.

In some embodiments, sensing elements 104 is non-symmetrical (e.g., parallelogram shaped) or angled to a lateral side of body 102, such as shown in FIG. 6B. Sensing element 104 being angled to a lateral side of body 102 (e.g., FIG. 6B) results in sensor 150 being sensitive to when deformation force Fis at an angle relative to longitudinal axis A-A. In some embodiments, sensing elements 104 are arranged in multiple shapes throughout sensor 150 such that sensing elements 104 (e.g., first set 104a and/or second set 104b) are sensitive when deformation force F is in multiple directions, such as in FIG. 6C. Sensing elements 104 may be substantially symmetrical about longitudinal axis A-A to allow sensing elements 104 to be sensitive to deformation force F when deformation force Fis at various angles relative to longitudinal axis A-A.

In some embodiments, sensing elements 104 are arranged in any combination of shape and/or configuration discussed herein and multiple sensors 150 with various arrangements of sensing elements 104, such as those discussed herein, are implanted into the patient to detect various movements of the spine. In some embodiments, multiple sensors 150 are disposed along or proximate to the spine of the patient. For example, more than one sensor 150 may be placed along the length of the spine to improve spatial resolution of the deformation of sensor 150. In some embodiments, multiple sensors 150 are used disposed on a shared substrate (e.g., substrate layer 110) to improve spatial resolution of local deformations on the spinal cord stimulation lead. Multiple sensor 150 may be disposed on the same substrate to form a unitary lead or sensor to improve spatial resolution of deformations of the spine. In some embodiments, multiple sensors 150 on a shared substrate are placed along the spine in multiple directions and positions. In some embodiments, multiple sensors 150 are placed along the spine in multiple directions and positions. In some embodiments, multiple sensors are placed adjacent to one another. Placing the sensors back-to-back may improve the special resolution of movements in multiple directions. Utilizing two sensors adjacent to each other enables the differentiation between movements in opposite directions. Extension and flexion of the spine are opposing movements and using sensors adjacent to each other permits differentiation between the movements. If a single sensor is used, the electrical resistance will appear similar between the movements of flexion and extension. By putting two sensors adjacent to each other the electrical resistance will be different between the sensors in flexion because the sensors will deform differently. The same will be true of extension.

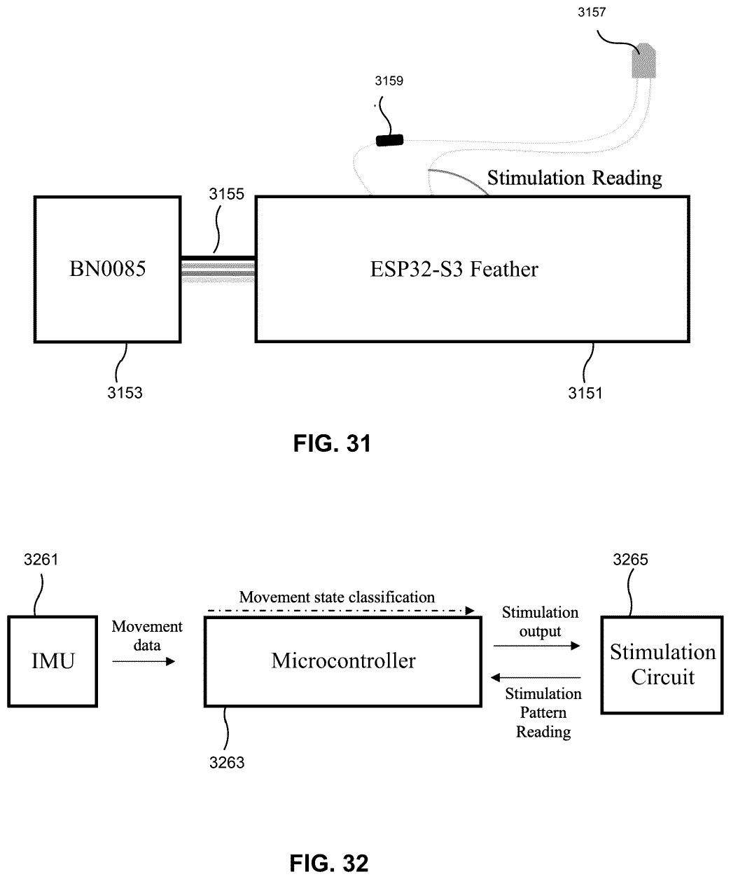

In embodiments, an external sensor (such as the Inertial Movement Units (IMU) depicted in FIGS. 31, 32, 33, and 35A-35I) is used to identify and measure movements of the neck and spine. Such sensor is typically worn on the head. For case of attachment the external sensor may be worn similar to a hearing aid. In some embodiments, the sensor is worn behind the auricle or outer ear. In some embodiments, the external sensor is worn within the auricle and held in place by the tragus and antitragus. In some embodiments, a portion of the sensor rests within the ear canal. The external sensor uses any or any combination of the following devices accelerometer, gyroscope, linear acceleration, gravity, rotation vectoring, vibration frequency. These devices detect movement such as tilt, rotation, swing, and shake. As the head moves, the sensor identifies and measures the movement and sends an electrical signal (i.e. an altered electrical property) indicating the direction and magnitude of the movement. This electrical property functions similarly to the electrical property of the implanted sensor. For example, in embodiments employing a sensor held behind the ear, the ear-worn sensor may have a MEMS based accelerometer, gyroscope, and magnetometer/compass integrated, and a microcontroller or other CPU capable of integrating data from all the sensors into an absolute orientation of the head. For example, an initial absolute orientation is an initial state and is associated with an initial electrical property (e.g. signal from the sensor or an associated controlling device). Movement into a new orientation results in altered state. The movement to, or the determination of an such an altered state results in in a change in sensor or controller transmitting an electrical signal, which, in this case, is the altered electrical property.

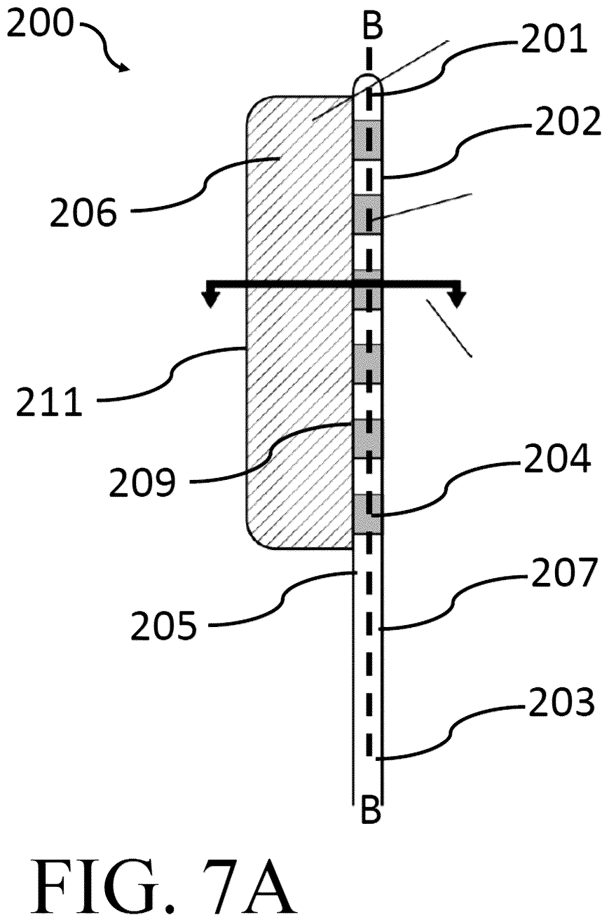

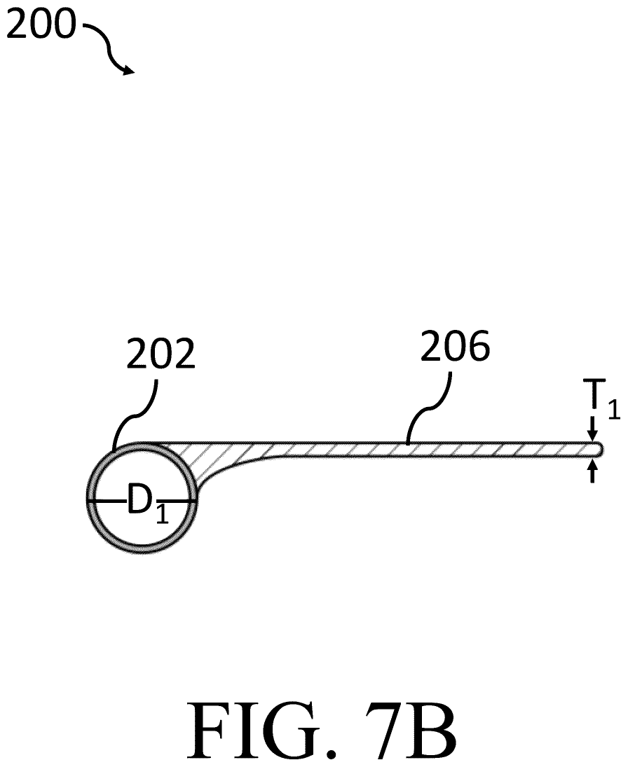

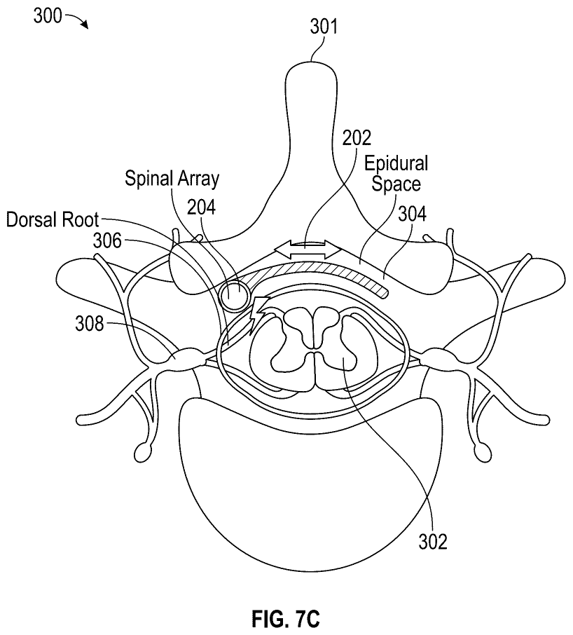

Referring to FIGS. 7A-13B, device 100 may include electrode 200. Electrode 200 may be implanted within the patient and may be in communication with neurostimulator 180. In some embodiments, electrode 200 is configured to provide electrical stimulation to a desired area. For example, electrode 200 may be implanted within a patient proximate their spine to deliver electrical stimulation to an area proximate their spine (e.g., dorsal roots). In use, electrode 200 may be implanted proximate the spinal cord and may include a stabilizing structure (e.g., stabilizing flap 206) configured to prevent movement of electrode 200. For example, movement of electrode 200 from its desired implanted location can result in inefficient or improper electrical stimulation being provide to the spine of the patient, in addition to being harmful to the patient. Precise placement of electrode 200 is desired to ensure that electrical stimulation is provided to the desired location. The stabilizing structure or flap may assist with enabling stabilizing electrode 200 within the spinal canal in addition allowing for precise placement of electrode 200 over the desired area targeted for stimulation (e.g., dorsal roots).

In some embodiments, electrical stimulation is provided to a desired area is to assist in muscle stimulation for the muscles of certain parts of the body. The electrical stimulation is to be provided responsive to the movement detected by the sensors. The sensors enable the direction and the magnitude of the movement to be measured. In some embodiments, the sensors and electrodes are implanted within the body. In particular embodiments, the electrical stimulation is modulated in response to the direction and/or magnitude of movement detected by the sensos. In more particular embodiments, the direction and/or magnitude of the movement alters the location and/or intensity of electrical stimulation and the electrical stimulation is modulated to compensate for the alteration of location and/or intensity. In most embodiments, the sensors and electrodes are implanted proximate to the spine. In some embodiments, the sensor is external and is worn on the body. In some of these embodiments, the sensor is worn on the head. In these embodiments, the electrode is implanted proximate to the spine. Once the direction and magnitude of the movement has been determined, the electrical stimulation provided to the appropriate area is modulated to compensate for the movement. In particular embodiments, the electrical stimulation may be modulated in a corresponding magnitude to assist in completing that movement.

In some embodiments, continuous electrical stimulation is provided. This continuous electrical stimulation assists in muscle stimulation and muscle movement. By providing continuous electrical stimulation, the amount of stimulation necessary to activate a muscle is lowered.

In embodiments, the modulation of the electrical stimulation is a reduction in stimulation by a particular electrode. In embodiments, the modulation of the electrical stimulation is increase in stimulation by a particular electrode. In further embodiments, the modulation of electrical stimulation is an increase in stimulation in at least a first electrode and a decrease in stimulation in at least a second electrode.

Referring to FIGS. 7A-7C, electrode 200 may include body 202 and a plurality of stimulating elements 204 disposed along a length of body 202 electrode 200. Body 202 may include first end 201 and second end 203, which may be opposite first end 201. Body 202 may be substantially linear and may include longitudinal axis B-B that extends along the length of body 202 and through first end 201 and second end 203. In some embodiments, stimulating elements 204 are disposed along body 202 between first end 201 and second end 203. For example, stimulating elements 204 may be disposed along longitudinal axis B-B. In some embodiments, body 202 is coupled to neurostimulator 180 and/or another implantable pulse generator (IPG) at, for example, second end 203. Body 202 may include first side 205 and second side 207. First side 205 may be opposite second side 207 and the width of body 202 may extend from first side 205 to second side 207.

In some embodiments, electrode 200 includes stabilizing flap 206. Stabilizing flap 206 may extend from body 202 at first side 205 and/or second side 207. In some embodiments, stabilizing flap 206 extends from body 202 between first end 201 and second end 203. Stabilizing flap 206 may extend laterally from body 202. During placement of electrode 200 within the epidural space spinal column, stabilizing flap 206 may abut the spinal cord or a structure proximate the spinal cord to prevent movement of electrode 200 relative to the spine. Further, stabilizing flap 206 may assist with placing body 202 and stimulating elements 204 lateral to the midline of the spine, such as proximate the dorsal roots. In some embodiments, placement of electrode 200 too close to the midline of the spine results in body 202 and stimulating elements 204 being too close to the midline of the spine. Placement of electrode 200 too medial or too lateral may impact the desired effects of the stimulation provided by electrode 200 to the desired area. Further, incorrect placement of electrode 200 may result in providing stimulation to an undesired area.

Stabilizing flap 206 may extend from body 202 between first end 201 and second end 203. However, stabilizing flap 206 may extend from any location of body 202. Stabilizing flap 206 may have a length less than or equal to the length of body 202. In some embodiments, stabilizing flap 206 is substantially rectangular. In alternative embodiments, stabilizing flap 206 is circular, triangular, hexagonal, oval, or any other shape desired. Stabilizing flap 206 may have smooth and/or curved corners to prevent inadvertent perforation or damage to the interior of the patient when electrode 200 is implanted within the patient, such as within the epidural space. Stabilizing flap 206 may be comprised of a biocompatible material and may be substantially flexible. In some embodiments, stabilizing flap 206 is substantially flexible while still having rigidity to provide structure to stabilizing flap 206.

In some embodiments, electrode 200 is comprised substantially similar to sensor 150. For example, electrode 200 may include one or more of substrate layer 110, resistive layer 112, and overmolding 114. In some embodiments, stabilizing flap 206 is comprised as an extension of overmolding 114. For example, during manufacturing of electrode 200, overmolding 114 may be expanded/stretched out to form stabilizing flap 206. In some embodiments, stabilizing flap 206 is formed via overmolding 114 and another formed on a portion of overmolding 114.

In some embodiments, electrode 200 is implanted into the patient via, for example, a laminectomy to expose the epidural space and allow for placement of electrode 200 within the epidural space. Electrode 200 may be inserted into the epidural space such that stabilizing flap 206 is positioned proximate the midline of the spine and body 202 and stimulating elements 204 are positioned lateral to the midline of the spine. For example, when electrode 200 is placed within the patient, stabilizing flap 206 may be configured to extend through the mid-sagittal plane of the patient. Once electrode 200 is inserted into the patient (e.g., within the epidural space), stabilizing flap 206 may be configured to conform to the shape of the epidural space and curve around the spinal cord. After implantation of electrode 200, stimulating elements 204 on body 202 may abut an area proximate the desired target area (e.g., dorsal roots). After implantation and alignment of body 202, stimulating elements 204, and stabilizing flap 206, electrode 200 may be restrained in place and movement of electrode 200 may be prevented due to stabilizing flap 206.

Referring to FIG. 7B, body 202 of electrode 200 may have maximum diameter or thickness D₁. For example, body 202 may be substantially cylindrical such that electrode 200 resembles a percutaneous electrode. In some embodiments, stabilizing flap 206 has maximum thickness T₁, which may be less than maximum diameter D₁ of body 202. For example, body 202 may be the thickest portion of electrode 200. In some embodiments, stabilizing flap 206 has maximum thickness T₁ that is substantially equal to maximum diameter D₁ of body 202. In alternative embodiments, stabilizing flap 206 has maximum thickness T that is greater than maximum diameter D₁ of body 202.

In some embodiments, stimulating elements 204 are disposed on body 202 proximate stabilizing flap 206. For example, stabilizing flap 206 may extend from body 202 at first side 205 and stimulating elements 204 may be longitudinally disposed on body 202 proximate first side 205. Stimulating elements 204 may be disposed on body 202 anywhere such as proximate second side 207 or distal to stabilizing flap 206. Body 202 and/or stabilizing flap 206 may include one or more stimulating elements 204. In some embodiments, stabilizing flap 206 extends from one or both of first side 205 and second side 207. For example, electrode 200 may include one or more stabilizing flaps 206 extending from first side 205 and/or may include one or more stabilizing flaps 206 extending from second side 207. In some embodiments, electrode 200 includes two or more stabilizing flaps 206 extending from first side 205 and/or second side 207. Each stabilizing flap 206 may be configured to move independently of the other stabilizing flap 206.

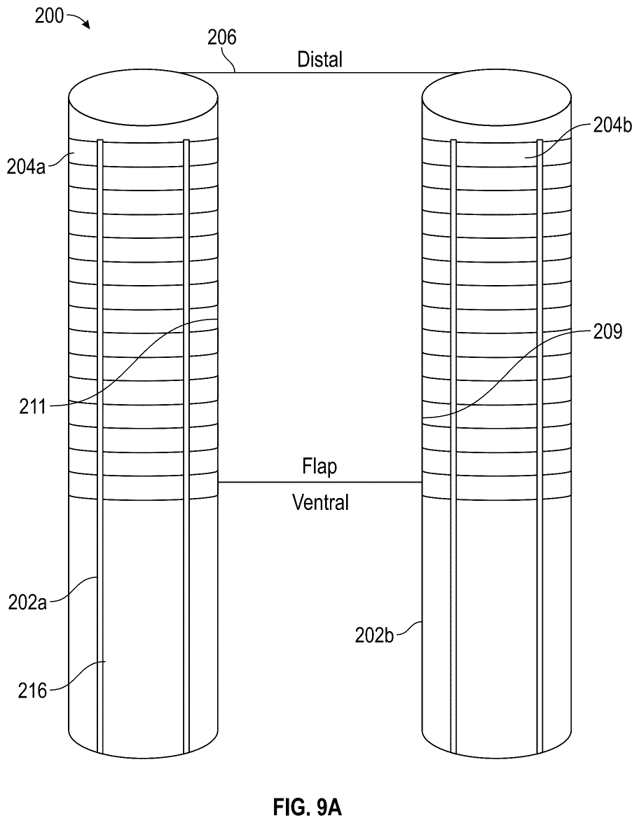

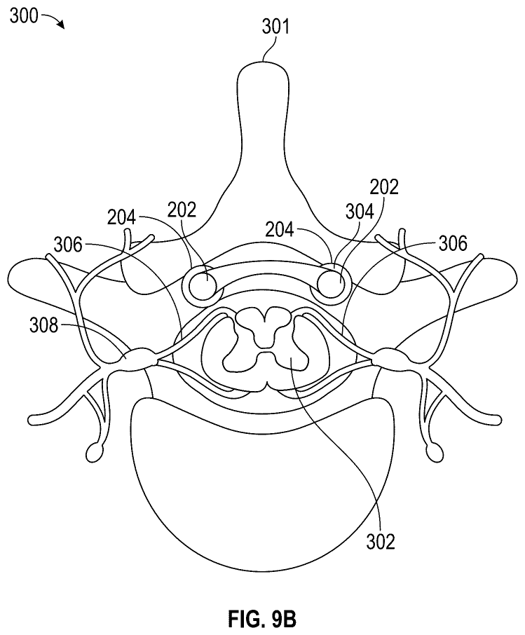

In some embodiments, stabilizing flap 206 includes first lateral end 209 and second lateral end 211, which may opposite first lateral end 209. Stabilizing flap 206 may have a width that extends from first lateral end 209 and second lateral end 211. Stabilizing flap 206 may be coupled to body 202 at first lateral end 209. In some embodiments, stabilizing flap 206 is coupled to one body 202 at first lateral end 209 and a second body 202 at second lateral end 211 (e.g., FIG. 9A). Each of the first body 202, the second body 202, and/or stabilizing flap 206 may have one or more stimulating elements 204.

In some embodiments, stabilizing flap 206 tapers from first lateral end 209 to second lateral end 211. For example, the thickness of stabilizing flap 206 may be greater proximate first lateral end 209 compared to second lateral end 211. In some embodiments, stabilizing flap 206 tapers from second lateral end 211 to first lateral end 209. In some embodiments, stabilizing flap 206 tapers from first lateral end 209 to an area between first lateral end 209 and second lateral end 211 and then increases in thickness proximate second lateral end 211.

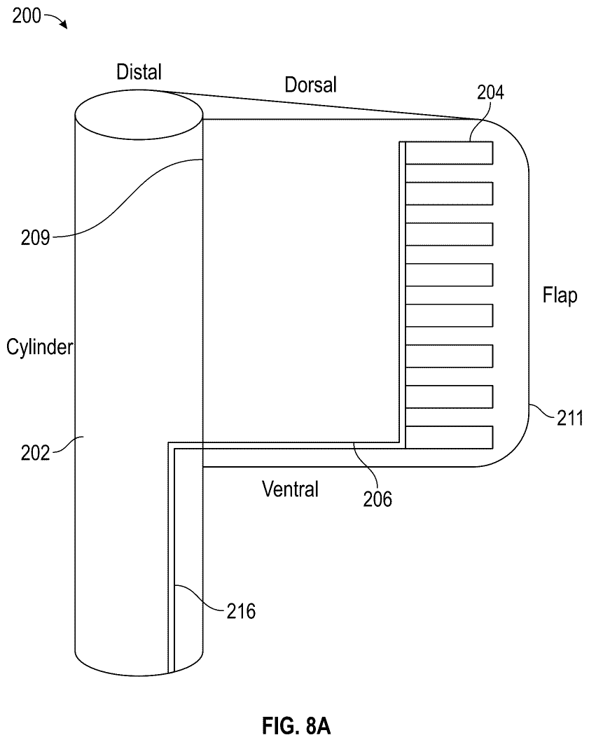

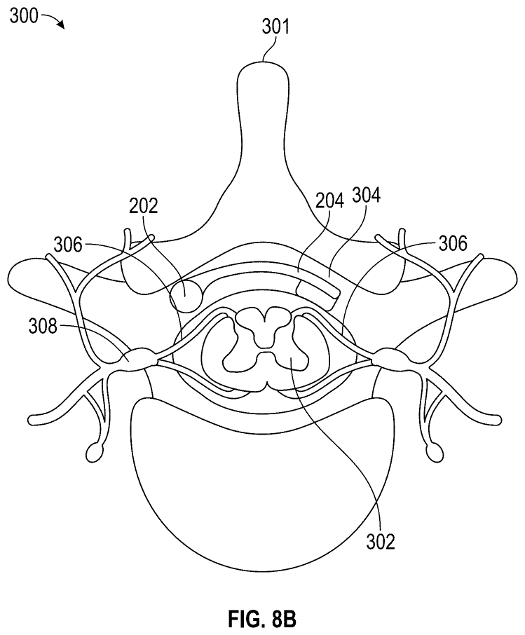

Referring to FIGS. 8A-14B, electrode 200 may have different configurations based on the placement and arrangement of stimulating elements 204, arrangement of stabilizing flap 206, placement of body 202 relative to stabilizing flap 206, and the number of bodies 202 of electrode 200. In some embodiments, stimulating elements 204 are coupled together via one or more wires 216. Wire 216 may be further coupled to neurostimulator 180 and/or an IPG. In some embodiments, wire 216 couples each stimulating element 204 to each other and to neurostimulator 180, a control circuit, and/or an IPG. In some embodiment, each electrode 200 is independently coupled to neurostimulator 180, a control circuit, and/or an IPG via its own wire 216. For example, each electrode 200 may have a corresponding wire 216 coupling electrode 200 to neurostimulator 180, a control circuit, and/or an IPG.

Referring to FIGS. 8A and 8B, stabilizing flap 206 may extend from body 202 and stabilizing flap 206 may include a plurality of stimulating elements 204. Stabilizing flap 206 may be coupled to body 202 at first lateral end 209 and a plurality of stimulating elements 204 may be disposed on stabilizing flap 206 proximate second lateral end 211.

As shown in FIG. 8B, electrode 200 in the configuration of FIG. 8A may be placed within vertebral body 301 of spinal column 300. For example, electrode 200 may be placed within epidural space 304 such that body 202 is proximate a first dorsal root 306 on a first side of spinal column 300 and stabilizing flap 206 containing stimulating elements 204 is disposed proximate a second dorsal root 306 on a second side of spinal column 300. In some embodiments, electrode 200 is disposed within spinal column 300 such that stimulating elements 204 provide electrical stimulation to one or more dorsal roots 306 without providing direct stimulation to dorsal root ganglion 308. Stabilizing flap 206 may contour to the shape of epidural space 304 such that stabilizing flap 206 bends around spinal cord 302.

Referring to FIGS. 9A and 9B, stabilizing flap 206 may extend from first body 202a and may couple to second body 202b. Each of first body 202a and second body 202b may include stimulating elements 204a and 204b respectively. Stabilizing flap 206 may be coupled to first body 202a at first lateral end 209 and second body 202b at second lateral end 211 proximate second lateral end 211.

As shown in FIG. 9B, electrode 200 in the configuration of FIG. 9A may be placed proximal to the spinal column 300. For example, electrode 200 may be placed within epidural space 304 such that first body 202a having stimulating elements 204a is proximate a first dorsal root 306 and second body 202b having stimulating elements 204b is proximate a second dorsal root 306. Stabilizing flap 206 may couple first body 202a to second body 202b and stabilizing flap 206 may be disposed within epidural space 304 proximate the midline of spinal cord 302 such that it contours within epidural space 304 proximate the midline of spinal cord 302.

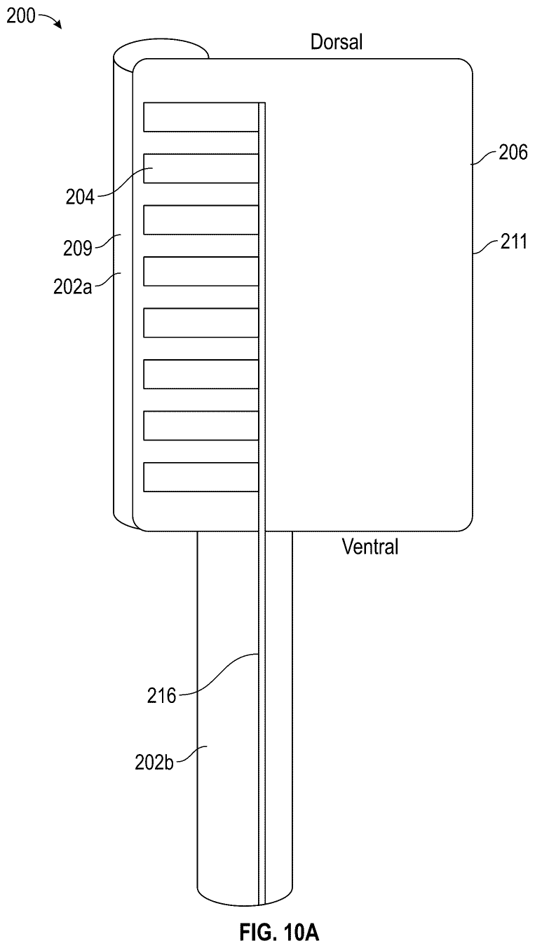

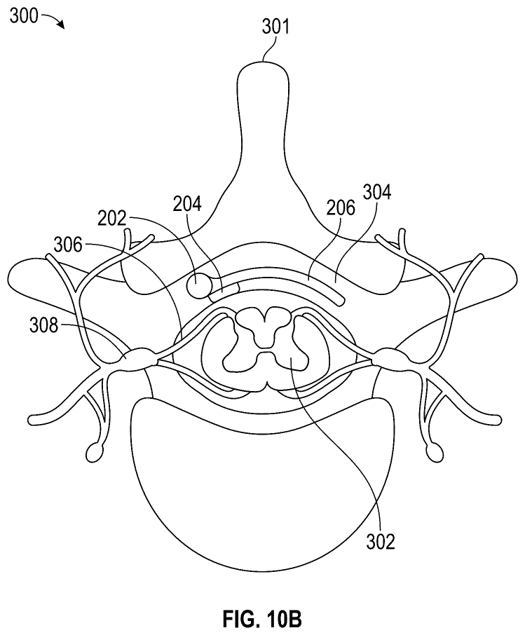

Referring to FIGS. 10A and 10B, stabilizing flap 206 may extend from first body 202a and stabilizing flap 206 may also be coupled to second body 202b, which may extend downward from stabilizing flap 206. For example, first body 202a may be coupled to stabilizing flap 206 proximate first lateral end 209 and stabilizing flap 206 may include stimulating elements 204 proximate first lateral end 209. First body 202a and stabilizing flap 206 may be coupled to second body 202b such that second body 202b extends downward from or proximate a midline of stabilizing flap 206.

As shown in FIG. 10B, electrode 200 in the configuration of FIG. 10A may be placed within vertebral body 301 of spinal column 300. For example, electrode 200 may be placed within epidural space 304 such that first body 202a and stabilizing flap 206 containing stimulating elements 204 are proximate dorsal root 306. Second body 202b may extend in the caudal direction away from first body 202a and stabilizing flap 206. In some embodiments, electrode 200 is disposed within spinal column 300 such that stimulating elements 204 provide electrical stimulation to one or more dorsal roots 306 without providing direct stimulation to dorsal columns. Stabilizing flap 206 may contour to epidural space 304 such that stabilizing flap 206 bends around spinal cord 302.

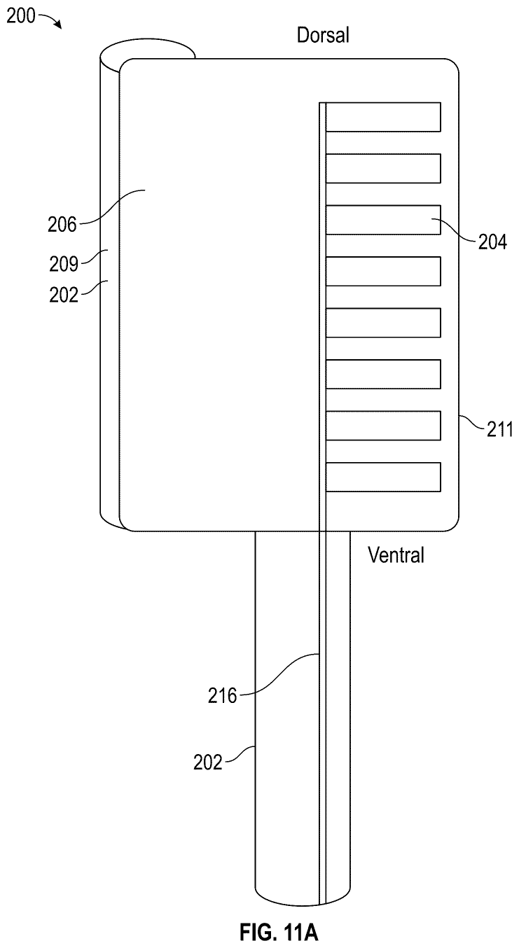

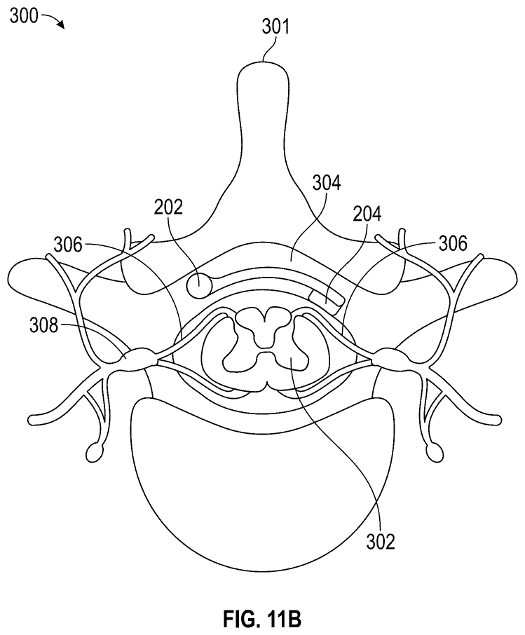

Referring to FIGS. 11A and 11B, stabilizing flap 206 may extend from first body 202a and stabilizing flap 206 may also be coupled to second body 202b, which may extend downward from stabilizing flap 206. First body 202a may be coupled to stabilizing flap 206 proximate first lateral end 209 and stabilizing flap 206 may include stimulating elements 204 proximate second lateral end 211. First body 202a and stabilizing flap 206 may be coupled to second body 202b such that second body 202b extends downward from stabilizing flap 206 from or proximate a midline of stabilizing flap 206.

As shown in FIG. 11B, electrode 200 in the configuration of FIG. 11A may be placed within vertebral body 301 of spinal column 300. For example, electrode 200 may be placed within epidural space 304 such that first body 202a is proximate a first dorsal root 306 and stabilizing flap 206 containing stimulating elements 204 are proximate a second dorsal root 306. Second body 202b may extend in the caudal direction away from first body 202a and stabilizing flap 206. In some embodiments, electrode 200 is disposed within spinal column 300 such that stimulating elements 204 provide electrical stimulation to one or more dorsal roots 306 without provide direct stimulation to dorsal root ganglion 308. Stabilizing flap 206 may contour to epidural space 304 such that stabilizing flap 206 bends around spinal cord 302.

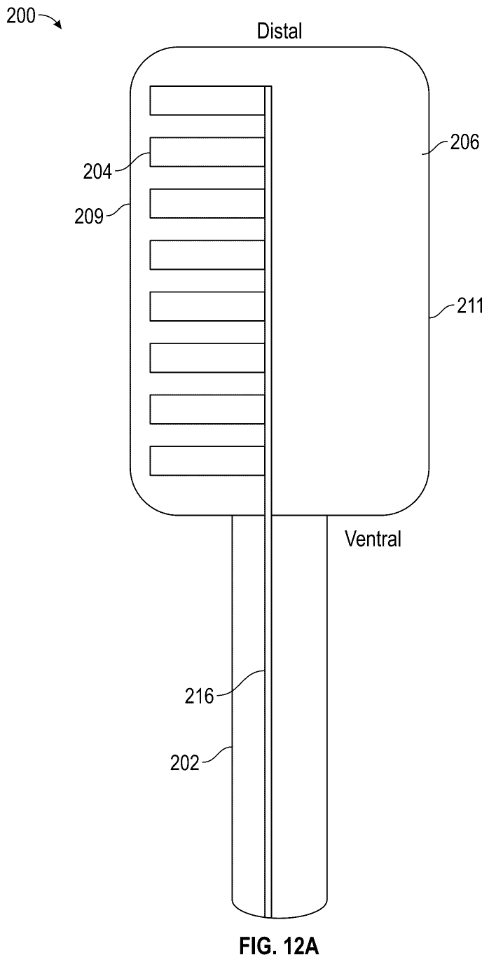

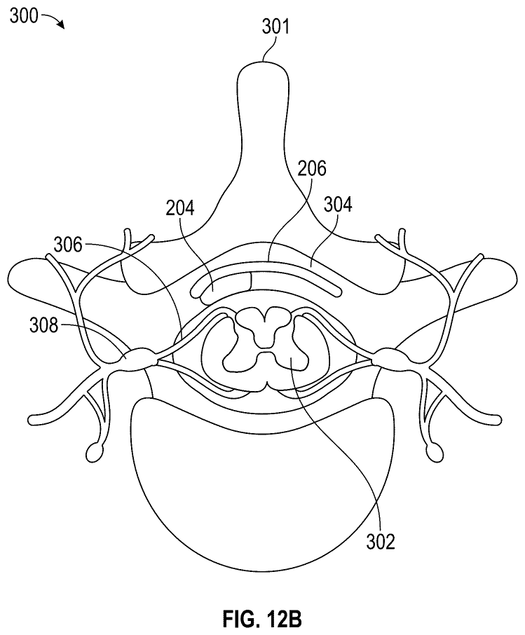

Referring to FIGS. 12A and 12B, stabilizing flap 206 may be coupled to body 202 and stabilizing flap 206 may contain stimulating elements 204 proximate first lateral end 209. Body 202 may be coupled to stabilizing flap 206 proximate the midline of stabilizing flap 206. In some embodiments, body 202 extends downward from stabilizing flap 206 from or proximate a midline of stabilizing flap 206. In alternative embodiments, stabilizing flap 206 includes stimulating elements 204 disposed proximate second lateral end 211.

As shown in FIG. 12B, electrode 200 in the configuration of FIG. 12A may be placed within vertebral body 301 of spinal column 300. For example, electrode 200 may be placed within epidural space 304 such that stabilizing flap 206 containing stimulating elements 204 are proximate dorsal root 306. In some embodiments, electrode 200 is disposed within spinal column 300 such that stimulating elements 204 provide electrical stimulation to one or more dorsal roots 306 without provide direct stimulation to dorsal root ganglion 308. Stabilizing flap 206 may contour to epidural space 304 such that stabilizing flap 206 bends around spinal cord 302.

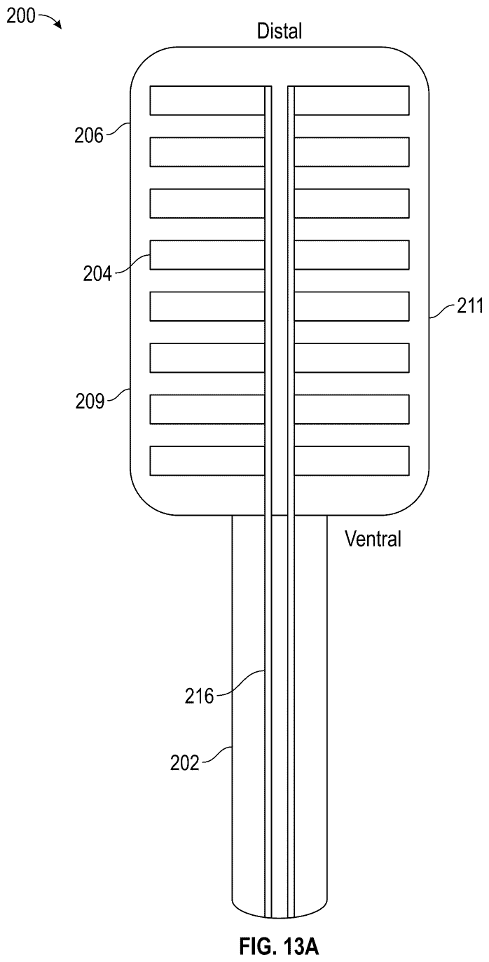

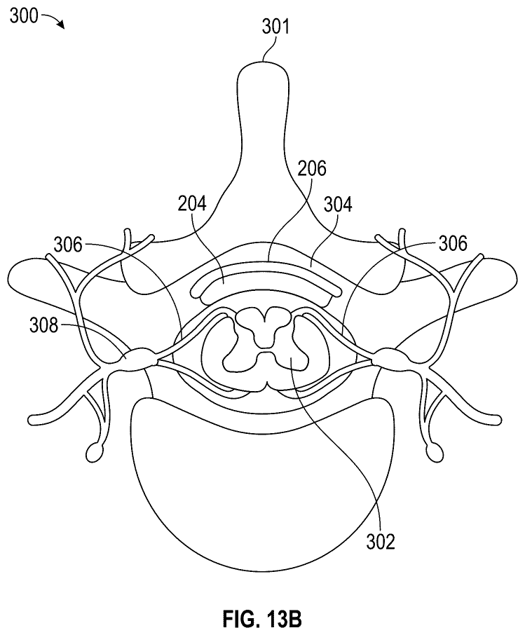

Referring to FIGS. 13A and 13B, stabilizing flap 206 may be coupled to body 202 and stabilizing flap 206 may contain stimulating elements 204 proximate first lateral end 209 and second lateral end 211. Body 202 may be coupled to stabilizing flap 206 proximate the midline of stabilizing flap 206. In some embodiments, body 202 extends downward from stabilizing flap 206 from or proximate a midline of stabilizing flap 206.

As shown in FIG. 13B, electrode 200 in the configuration of FIG. 13A may be placed within vertebral body 301 of spinal column 300. For example, electrode 200 may be placed within epidural space 304 such that stabilizing flap 206 containing stimulating elements 204 are proximate dorsal root 306. Stimulating elements 204 disposed on stabilizing flap 206 may provide stimulation to an area proximate a first dorsal root 306 to an area proximate a second dorsal root 306. For example, stabilizing flap 206 may extend from one dorsal root 306 lateral to the midline of spinal cord 302 to another dorsal root 306 on the other side of the midline of spinal cord 302. In some embodiments, stimulating elements 204 is configured to provide stimulation to the area covered by stabilizing flap 206 (e.g., the area between dorsal roots 306 extending through the midline of spinal column 300). In some embodiments, electrode 200 is disposed within spinal column 300 such that stimulating elements 204 provide electrical stimulation to one or more dorsal roots 306 without provide direct stimulation to dorsal root ganglion 308. Stabilizing flap 206 may contour to epidural space 304 such that stabilizing flap 206 bends around spinal cord 302.

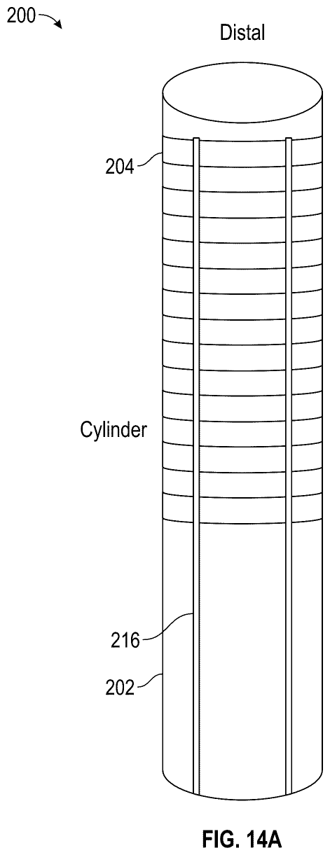

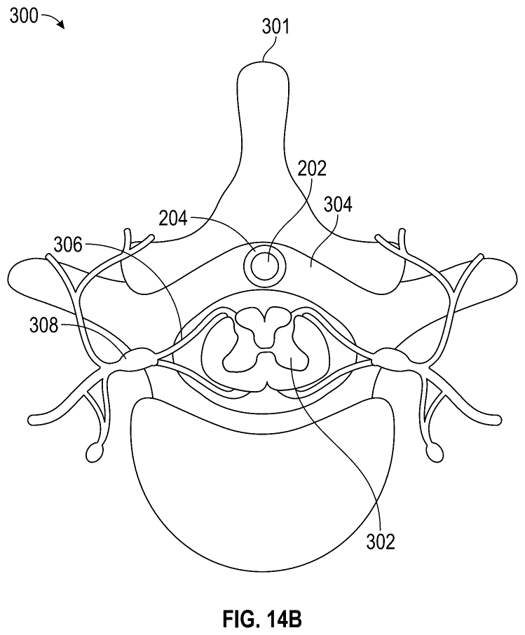

Referring to FIGS. 14A and 14B, electrode 200 may not include stabilizing flap 206. Electrode 200 may include body 202, which may be substantially cylindrical. Body 202 may include stimulating elements 204 extending the length of body 202.

As shown in FIG. 14B, electrode 200 in the configuration of FIG. 14A may be placed within vertebral body 301 of spinal column 300. For example, electrode 200 may be placed within epidural space 304 proximate the midline of spinal cord 302. Stimulating elements 204 disposed on body 202 may provide stimulation to an area proximate the midline of spinal cord 302. In some embodiments, electrode 200 is disposed within spinal column 300 such that stimulating elements 204 provide electrical stimulation to an are proximate one or more dorsal roots 306 without provide direct stimulation to dorsal root ganglion 308.

In some embodiments, the features of electrode 200 disclosed herein and in FIGS. 7A-14A are incorporated into sensor 150 and the features of sensor 150 disclosed herein and in FIGS. 2A-6C are incorporated into electrode 200. For example, sensor 150 may include one or more stimulating elements 204 and electrode 200 may include one or more sensing element 104. In some embodiments, sensor 150 includes stabilizing flap 206. For example, body 102 of sensor 150 may include stabilizing flap 206 extending from one or more lateral sides of body 102. In some embodiments, stimulating elements 204 and sensing elements 104 are interchangeable with each other in both sensor 150 and electrode 200. For example, sensor 150 may include one or more of sensing elements 104 and stimulating elements 204 and electrode 200 may include one or more of sensing elements 104 and stimulating elements 204.

In embodiments, the sensing unit is an external unit such as an Inertial Movement Unit (IMU). In some embodiments, the IMU is connected to and communicates with an implanted unit using wires. The implanted unit may include sensors and may include electrodes. In some embodiments, the IMU communicates with the implanted unit wirelessly. Such wireless communication includes but is not limited to Bluetooth. Near Field Communication (NFC), or radio frequency communication.

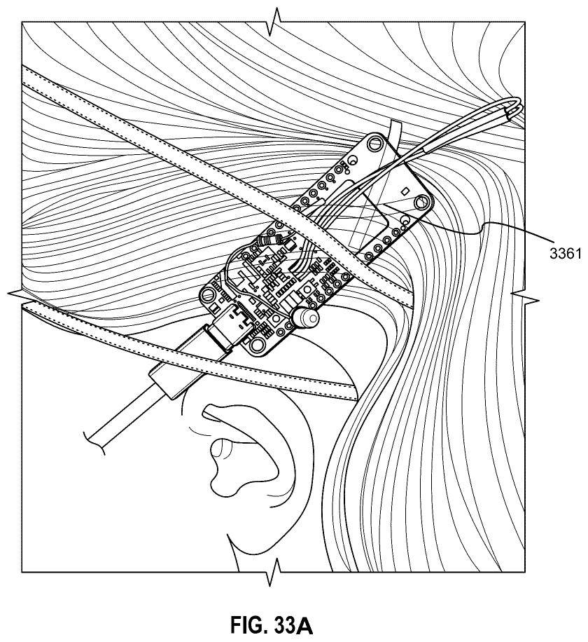

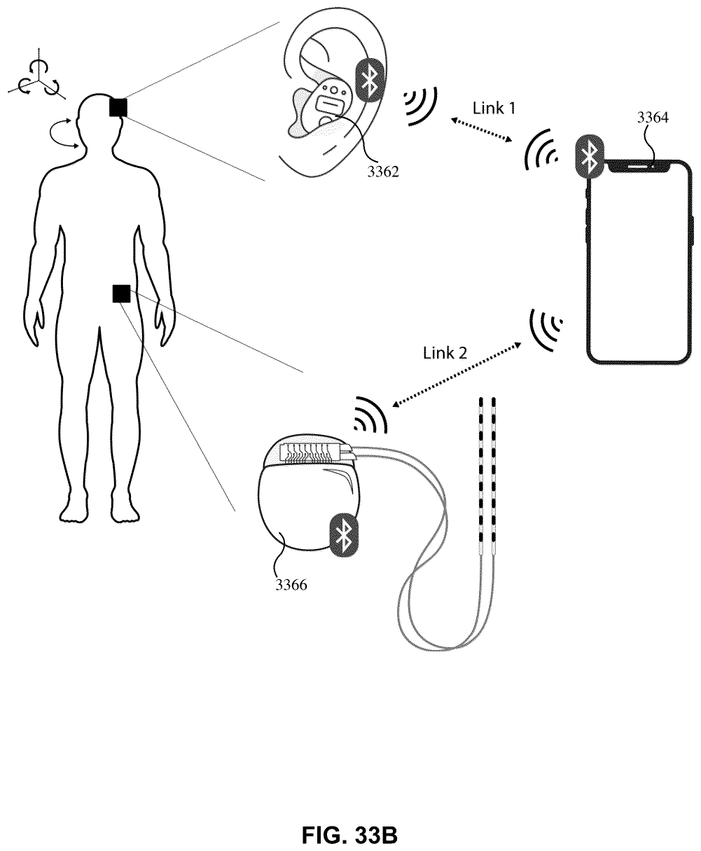

FIG. 33B is a depiction of an in ear IMU 3362 wirelessly connected with a controller, which in this embodiment is a smart phone 3364. The Smart phone 3364 is in turn wirelessly connected to the implanted unit 3366. This embodiment depicts Bluetooth as the wireless communication method.

In some embodiments, the orientation information is sent via Bluetooth to a controller such as a patient's phone. The controller will generate a set of commands to change the stimulation accordingly that would be sent via a wireless link to the implant. Absolute orientation is not the only valuable signal, movement in any of the 9 degrees of freedom axes is useful on its own to detect instantaneous movement. There is value in recording sensor values directly as well as the processed orientation that is output by the IMU. The commands sent to the stimulator from the controller or smart phone could either be a new set of stimulation parameters that should be set (for example ‘decrease amplitude to 3 mA’), or it could be a command to switch to a pre-programmed configuration when certain head movements are detected. Pre-programmed configurations require less data bandwidth and are likely safer because there is no chance for the stimulator to be put into a state other than those pre-programmed into the device. Either method could be made safe if needed. While the movement sensor does not need to update as quickly as the implanted sensors, it would need to be able to update stimulation on the order of <50 ms, to adapt quickly enough to be effective when a patient moves their head. The controller or programmer could be any device capable of sending update commands to the stimulator. In some embodiments, a patient's own phone running an application, will connect via Bluetooth to both the ear-worn sensor and the implanted stimulator. The controller, such as a patient's phone receives orientation updates and send stimulation changes to the stimulator accordingly.

### Examples

The foregoing descriptions have explained the devices and the procedures by which sensors are used to measure deformation and increase stimulation in a patient.

### Flex Sensor Stimulation

This example depicts a model of flex sensors as they would function in use. The flex sensors are placed in a cervical laminectomy model, as the sensor would be implanted in a patient. The model is flexed and extended as would occur in a human cervical spine, to show the use of the sensing and stimulation components of the sensor as they work in concert in a closed loop system to modulate stimulation of the stimulation sensor in response to the sensor value of the sensor component.

Two mechanisms of stimulation are being used. The programing for stimulation is being directed by two closed loop algorithms. The first algorithm directs the sensors to adjust stimulation when head movements detected by the sensing component exceed a specific threshold. The second algorithm directs the sensors to adjust the stimulation continuously and proportionally to the detected head movement. The following components were used in the development of the circuitry for this sensor device;
Adafruit Metro
Adafruit Flex Sensors (2)
240Ω Resistor
120 KΩ Resistors (2)
LED

FIG. 15 depicts the physical setup of the sensor. A microcontroller 1531, in this case an Adafruit Metro microcontroller, was used to measure head movement using 2 flex sensors 1533a and 1533b, Adafruit flex sensors, constructed similar to the “resistive” sensors described above. Two sensors used together to distinguish between flexion, extension, and torsion. The microcontroller interpreted the sensor data and changed the frequency of an output square waveform that could be used to trigger spinal stimulation. The output waveform was visualized using an LED, and digitally captured by the microcontroller for review.

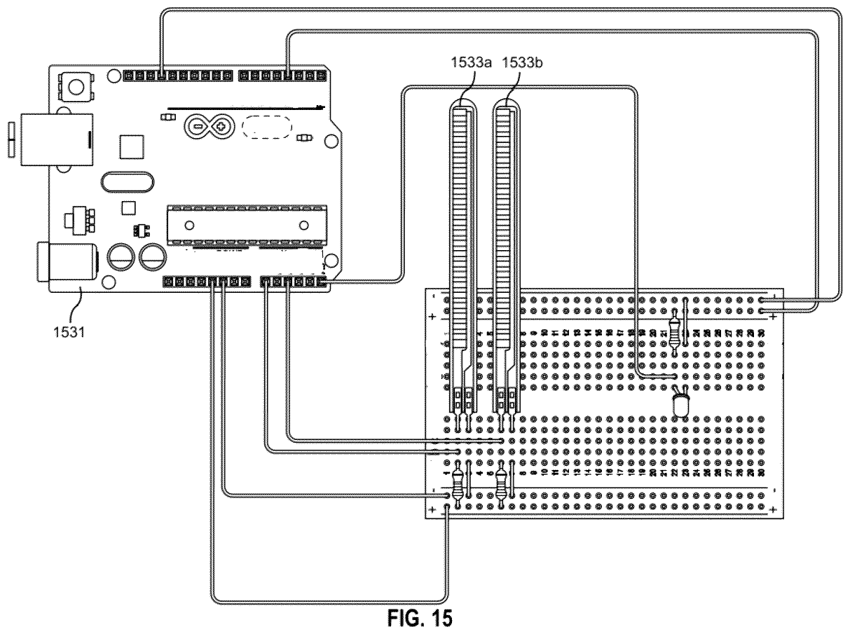

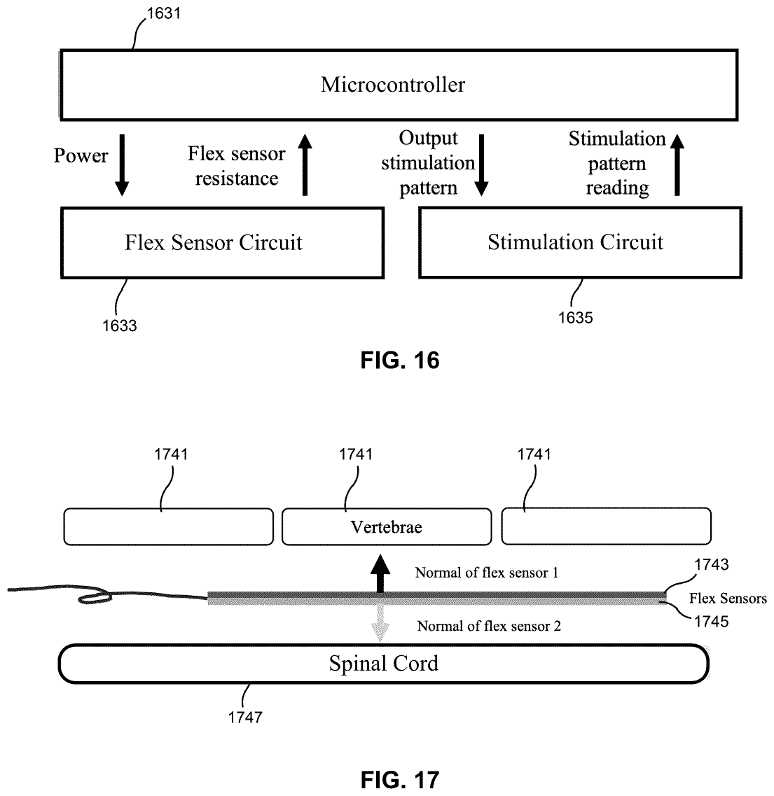

FIG. 16 is a flow chart of the communication flow. Microcontroller 1631 powers the flex sensor circuit 1633. The flex sensor circuit 1633 passes the value of the measured resistance to the microcontroller 1631. The algorithm stored in the processor of the microcontroller reads the value of the measured resistance and outputs a stimulation pattern to the stimulation circuit 1635. The stimulation circuit 1635 stimulates the nerves. A reading of the stimulation pattern is sent to the microcontroller.

FIG. 17 is a simplified side cutaway view of the sensors as they would be inserted. The sensors are inserted between the vertebrae 1741 and the spinal cord 1747. Two sensors are placed back-to-back. The flex sensor 1743 is located adjacent to the vertebrae 1741. Flex sensor 1745 is located adjacent to the spinal cord 1747. In some embodiments, the flex sensor 1745 adjacent to the spinal cord includes an electrode to provide stimulation.

Setting up the test proceeds along the following steps
1. Upload “flex_threshold.ino” to the Adafruit Metro
2. Place the flex sensors in midline the spinal column of cervical laminectomy model so that the bottom of the flex sensor rests at the bottom of the last vertebrae on the model.
3. Situate the model with sensors in place on ruled cutting mat with the bottom of the sensors and the last vertebrae aligned with the 3″ line on the horizontal plane of the mat.
4. In an excel sheet, record the starting posterior and anterior positions of the C2 vertebrae and the vertical length between the top of the C2 spinous process and the bottom of the last vertebrae.
5. Record the horizontal distance from the bottom edge of the system to the apex of the resting spinal curvature.
6. Open the CoolTerm application and connect it to the serial port that the Metro is connected to then start the recording to save the serial data as a .txt file.
7. Holding the bottom of the system in place at the 3″ on the mat, push the top of the model until the C2 spinous process is displaced 1.5″. This is one rep of extension.
8. Return the model to the original position.
9. Holding the bottom of the model in place, pull the top of the model back until the anterior end of the C2 vertebrae is displaced 1″ horizontally. This is one rep of flexion.
10. Repeat steps 8-10 five times.
11. In the Cool Term software, stop recording.
12. Open a new window in excel and select open in the file settings.
13. Select the file you generated in CoolTerm and go to next.
14. Select commas as the delineator.
15. Open the file and resave as a csv.
16. Use the recorded data to produce plots.

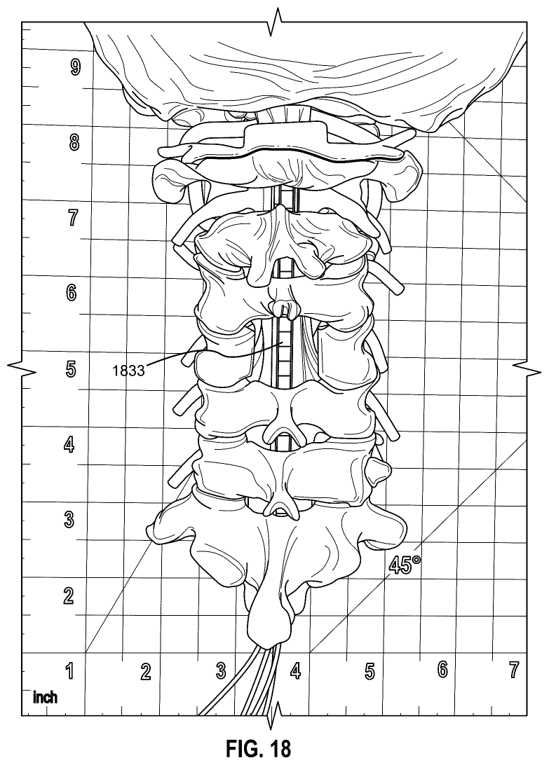

FIG. 18 depicts a dorsal view of the test set-up. A laminectomy model of the cervical spine is prepared with the flex sensors 1833 inserted in place between the vertebrae and the spinal cord.

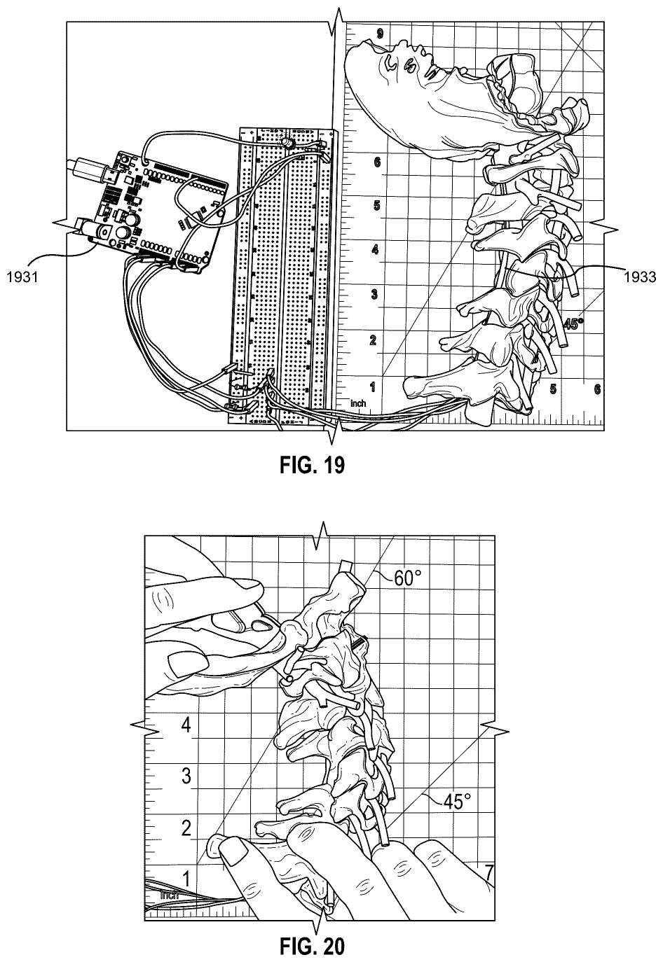

FIG. 19 depicts a lateral view of the test set-up. The flex sensors 1933 are connected to a microcontroller 1931.

FIG. 20 depicts a lateral view of the test set-up, with the model in a flexed position. The base of the model is held in a fixed or constant position and the top of the model moved in a manner that simulates flexion of the cervical vertebrae. It should be noted that the flexion of the cervical spine for this test is relative to the backward or dorsal direction of the spine as it is positioned in the body. In actual use, the flexion of the cervical spine would cause the head to lift. This can be visualized by lifting the chin.

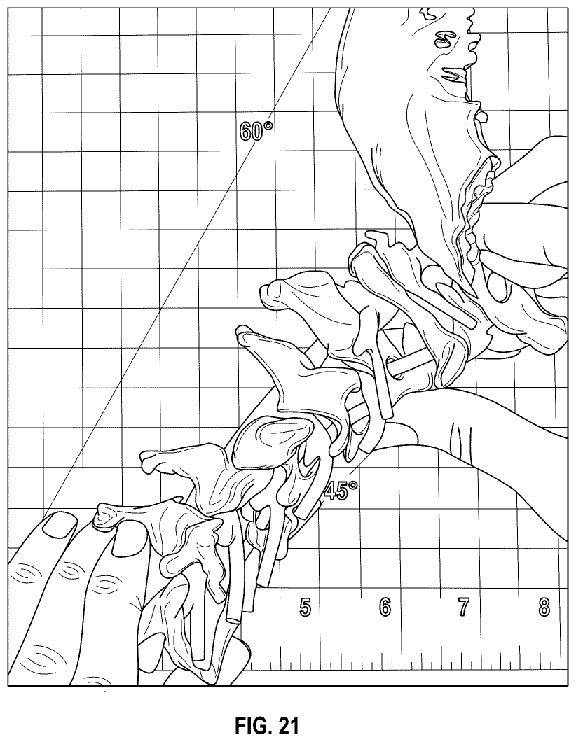

FIG. 21 depicts a lateral view of the model with the model in an extension position. The base of the model is held in a fixed or constant position, and the top of the model is moved in a manner that simulates extension of the cervical vertebrae. It should be noted that the extension of the cervical spine for this test is relative to the forward or ventral direction of the spine as it is positioned in the body. In actual movement of the cervical spine as a portion of the body, extension of the cervical spine would cause the head to lower. This can be visualized by lowering the chin.

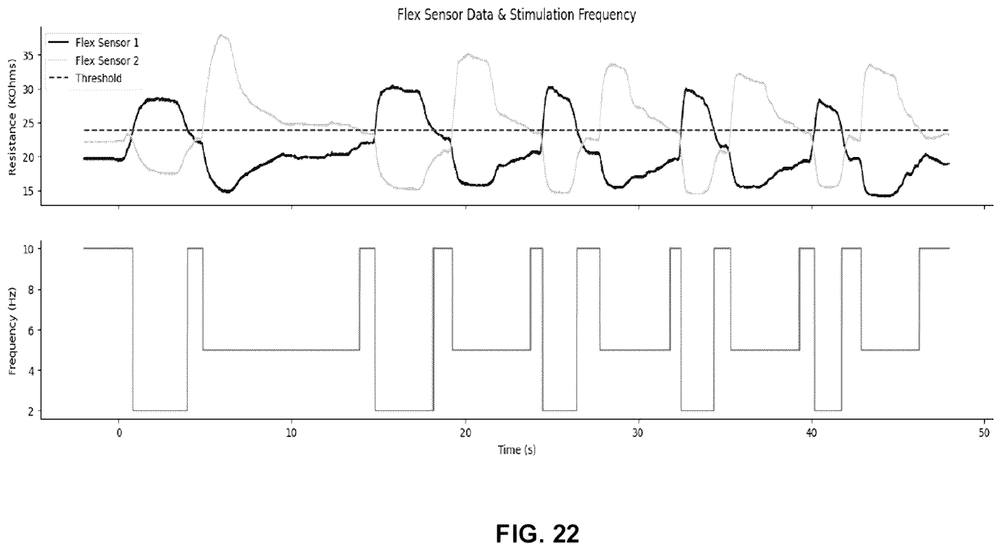

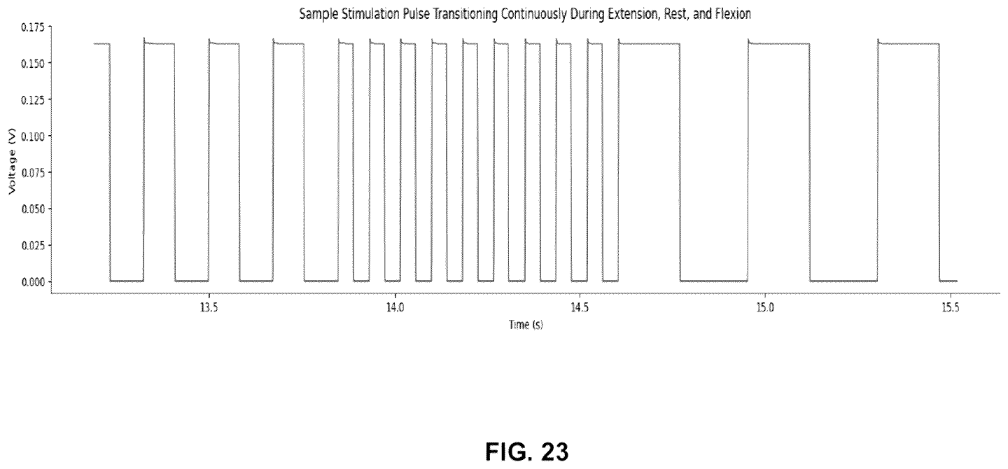

This testing setup was used to verify the procedure. Resistance thresholds were determined empirically based on a calibration phase where the head model was moved to observe the resulting resistance changes of each sensor. The threshold was selected to represent a moderate amount of extension or flexion, representative of the level of movement expected to yield user-perceived differences in electrical stimulation. A threshold value of 23.6 KΩ on flex sensor 1 represented a deflection in the “extension direction”, while the same threshold applied to flex sensor 2 represented a deflection in the “flexion” direction. At rest, the stimulation is at a frequency of 10 Hz. When extension is detected the frequency drops to 2 Hz, and in flexion the frequency changes to 5 Hz. FIG. 22 depicts the flex sensor values during the testing procedure, and the threshold used to determine the position. Flexion periods are highlighted in light gray and the extension periods are highlighted in dark gray. FIG. 23 depicts time window, from about 10 seconds to about 20 seconds, of the stimulation pulse during one transition between extension and flexion. Flexion periods are again highlighted in light gray and extension periods in dark gray. The non-highlighted section shows the higher frequency rest stimulation. As can be seen in these figures, as the resistance in each of the flex sensors crosses the threshold values, the stimulation pulse changes accordingly.

This test verifies the efficacy of changes in stimulation based on a threshold of flex sensor values. Throughout the test, the flex sensor values fluctuate as the model switched between flexion and extension. When the flex sensor 1 resistance crosses the threshold (FIG. 22), the extension state is detected, and the stimulation frequency changes accordingly. Similarly, flexion is detected when flex sensor 2's resistance crosses the threshold, and the resulting frequency change is observed. These results are further validated by the stimulation pulse shown in FIG. 23 where the appropriate changes in stimulation frequency occur with state changes.

Threshold Based Stimulation Modulation with Torsion

Limitations of the sensors did not allow detection of a torsion state in the spinal model. As a result, the flex sensors were removed from the laminectomy model and tested by hand with the following procedure to show proof of concept torsion state detection. In future applications, alternative or custom developed sensors may be employed to detect torsion with the sensors in place.
1. Upload “flex_threshold.ino” to the Adafruit Metro
2. Hold the flex sensors fixed at their base.
3. Situate the sensors in place on ruled cutting mat with the bottom of the sensors aligned with the 3″ line on the horizontal plane of the mat.
4. Open the CoolTerm application and connect it to the serial port that the Metro is connected to then start the recording to save the serial data as a .txt file.
5. Holding the bottom of the system in place at the 3″ on the mat, push the top of the model until the top of the sensors are displaced 1″. This is one rep of extension.
6. Return the sensors to the original position.
7. Holding the bottom of the model in place, pull the top of the sensors back until the tip is displaced 1″ horizontally. This is one rep of flexion.
8. Return the sensors to the original position.
9. Still holding the base, pinch the top of the sensors between your thumb and pointer finger and rotate your hand counterclockwise.
10. Return the sensors to the original position.
11. Still holding the base, pinch the top of the sensors between your thumb and pointer finger and rotate your hand clockwise.
12. Repeat steps 5-11 three times.
13. In the Cool Term software, stop recording.
14. Open a new window in excel and select open in the file settings.
15. Select the file you generated in CoolTerm and go to next.
16. Select commas as the delineator.
17. Open the file and resave as a csv.
18. Use the recorded data to produce plots.

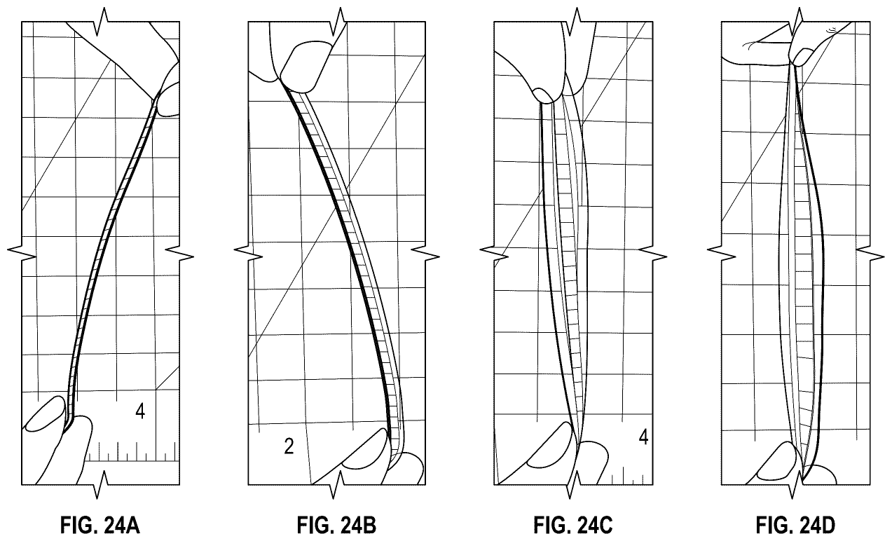

FIGS. 24A-24D depict the flex sensors in various states movement. As the laminectomy model was not suited for use in testing the effect of torsion on the flex sensors, tests were run with the sensors removed from the model. The sensors were once again paired with two sensors back-to-back. FIG. 24A depicts the sensor in position to simulate extension. FIG. 24B shows the sensor in position to simulate flexion. The positions and movement of the sensors has been maintained relative to the simulated positions used with the laminectomy model, that is, the flexion position of the sensors when out of the model mimics the flexion position of the sensors in the laminectomy model. The same is true for the extension position of the sensors. FIG. 24C depicts one instance of torsion. FIG. 24D depicts a second instance of torsion.

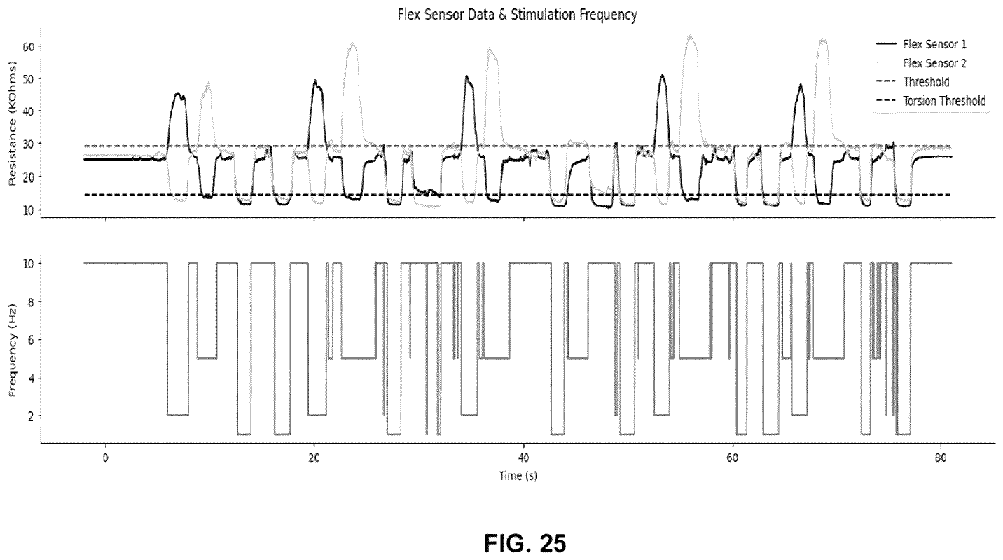

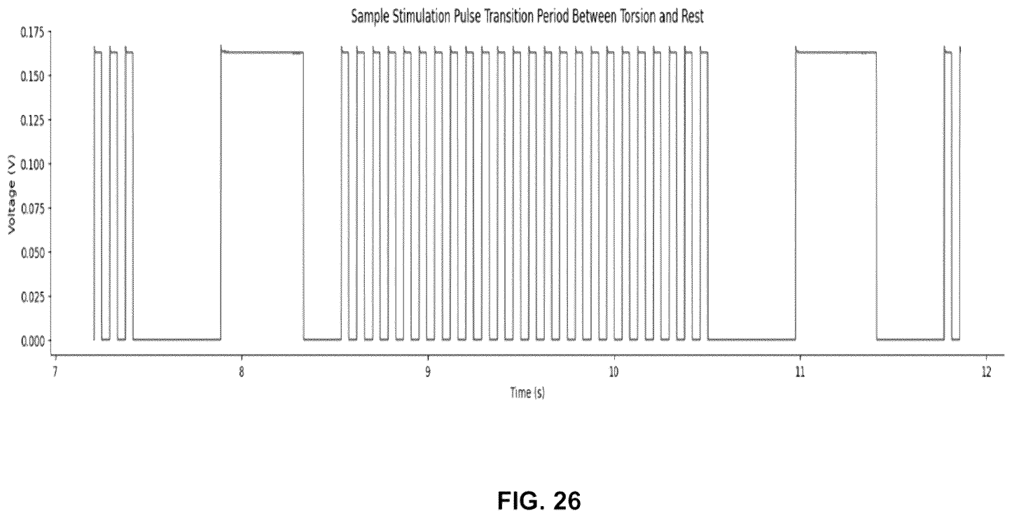

The data collected from the torsion testing procedure is depicted in FIGS. 25 and 26. Resistance thresholds were determined empirically based on a calibration phase where the flex sensors were moved to observe the resulting resistance changes of each sensor. A threshold value of 29.1 KΩ on flex sensor 1 represented a deflection in the “extension direction”, while the same threshold applied to flex sensor 2 represented a deflection in the “flexion” direction. The Torsion threshold was selected via the same empirical calibration phase. Deflection classified as “torsion” was achieved when flex sensor 1 and 2 dropped below the 14.4 KΩ torsion threshold. At rest, the stimulation is at a frequency of 10 Hz. When extension is detected the frequency drops to 2 Hz, in flexion the frequency changes to 5 Hz, and torsion results in a 1 Hz stimulation. FIG. 25 depicts the flex sensor values. Flexion periods are highlighted in light gray and extension periods are highlighted dark gray. Detected torsion is indicated by the lightest shading. Torsion is detected when both sensor values are below the Torsion Threshold. FIG. 26 shows a 6 second time window of the stimulation pulse during one transition between rest and both directions of torsion. The detected torsion periods are highlighted in very light grey. The segment without highlighting reflects the rest stimulation with higher frequency.

### Continuous Stimulation Modulation

In some embodiments of the flex sensors, it is advantageous to continuously provide stimulation to the spinal cord or an area near the spinal cord. Continuous stimulation of the spinal cord or the area near the spinal cord reduces the added stimulation necessary to affect a response. It may be beneficial to think of the continuous stimulation as lowering the activation energy of the neuromuscular response.
1. Upload “flex_diff_2sensors.ino” to the Adafruit Metro
2. Place the flex sensors in midline the spinal column of cervical laminectomy model so that the bottom of the flex sensor rests at the bottom of the last vertebrae on the model.
3. Situate the model with sensors in place on ruled cutting mat with the bottom of the sensors and the last vertebrae aligned with the 3″ line on the horizontal plane of the mat.
4. In an excel sheet, record the starting posterior and anterior positions of the C2 vertebrae and the vertical length between the top of the C2 spinous process and the bottom of the last vertebrae.
5. Record the horizontal distance from the bottom edge of the system to the apex of the resting spinal curvature.
6. Open the CoolTerm application and connect it to the serial port that the Metro is connected to then start the recording to save the serial data as a .txt file.
7. Holding the bottom of the system in place at the 3″ on the mat, push the top of the model until the C2 spinous process is displaced 1.5″. This is one rep of extension.
8. Return the model to the original position.
9. Holding the bottom of the model in place, pull the top of the model back until the anterior end of the C2 vertebrae is displaced 1″ horizontally. This is one rep of flexion.
10. Repeat steps 8-10 five times.
11. In the Cool Term software, stop recording.
12. Open a new window in excel and select open in the file settings.
13. Select the file you generated in CoolTerm and go to next.
14. Select commas as the delineator.
15. Open the file and resave as a csv.
16. Use the recorded data to produce plots.

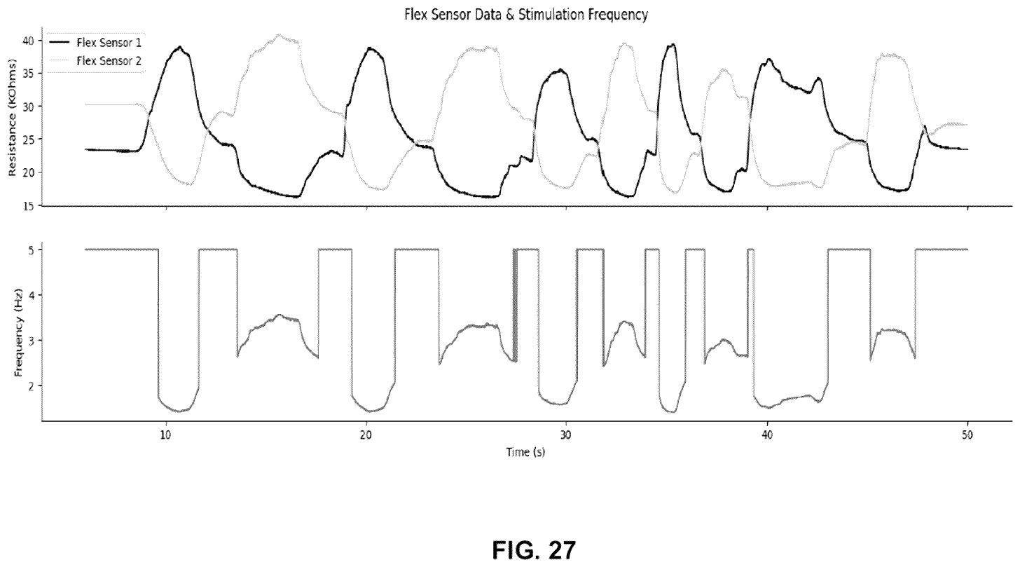

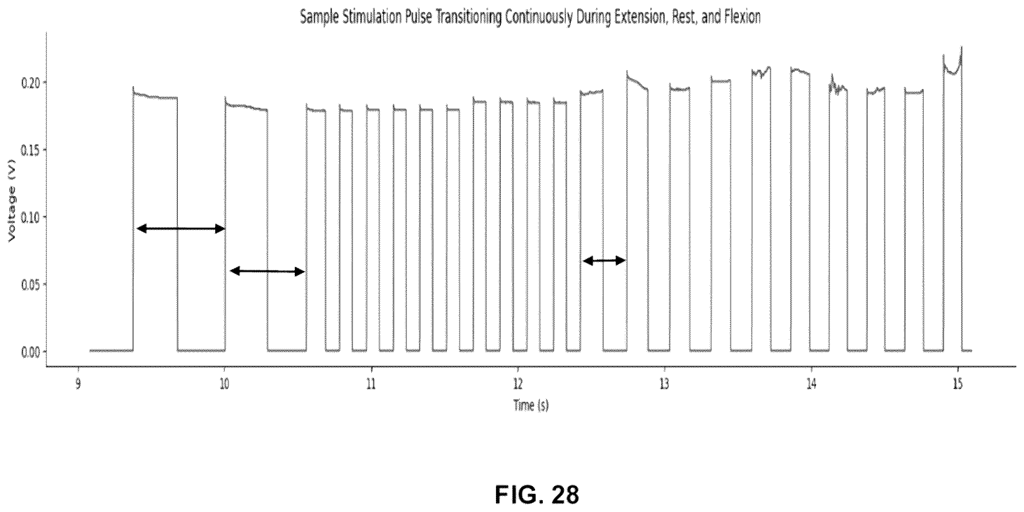

FIG. 27 depicts the data collected from the above testing procedure. The deflection state in this test was determined by a threshold on the difference between flex sensor resistances. This threshold was determined empirically based on a calibration phase where the flex sensors were moved to observe the resulting resistance changes of each sensor. When flex sensor 1's resistance exceeded that of flex sensor 2 by at least 2.56 KΩ, the deflection state was “extension.” If flex sensor 2's resistance was at least 2.56 KΩ greater than that of flex sensor 1, the deflection state was “flexion.” Once a new deflection state was detected, the larger flex sensor resistance value was used to modulate the stimulation pulse continuously and proportionally to that sensor reading. At rest the stimulation had a 5 Hz frequency. Upon detecting the extension state, the base frequency would drop to 2 Hz and continuously decrease with the extension movement. When flexion was detected, the base stimulation frequency would change to 3 Hz and continuously increase with the flexion movement. FIG. 27 depicts flex sensor values during our testing procedure and highlighted regions to indicate the state. Flexion is indicated by dark grey highlighting and extension is shown in light grey. FIG. 28 depicts a zoomed in 6 second window of the stimulation pulse during a change between flexion and extension. The solid arrows display the period of a higher frequency pulse. These arrows are overlaid on another pulse with an underlying dashed black arrow that shows the period of this pulse. This captures the differences in the stimulation pulse frequency as the model flexes or extends. This data shows the efficacy of a continuous change in stimulation output based on a difference between the flex sensor value and the detection threshold previously described.

Continuous Stimulation Modulation with Torsion

Limitations of the sensors did not allow detection of a torsion state in the spinal model. As a result, the flex sensors were removed from the laminectomy model and tested by hand with the following procedure to show proof of concept torsion state detection. In future applications, alternative or custom developed sensors may be employed to detect torsion with the sensors in place.
1. Upload “flex_diff_2sensors.ino” to the Adafruit Metro
2. Hold the flex sensors fixed at their base.
3. Situate the sensors in place on ruled cutting mat with the bottom of the sensors aligned with the 3″ line on the horizontal plane of the mat.
4. Open the CoolTerm application and connect it to the serial port that the Metro is connected to then start the recording to save the serial data as a .txt file.
5. Holding the bottom of the system in place at the 3″ on the mat, push the top of the model until the top of the sensors are displaced 1″. This is one rep of extension.
6. Return the sensors to the original position.
7. Holding the bottom of the model in place, pull the top of the sensors back until the tip is displaced 1″ horizontally. This is one rep of flexion.
8. Return the sensors to the original position.
9. Still holding the base, pinch the top of the sensors between your thumb and pointer finger and rotate your hand counterclockwise.
10. Return the sensors to the original position.
11. Still holding the base, pinch the top of the sensors between your thumb and pointer finger and rotate your hand clockwise.
12. Repeat steps 5-11 three times.
13. In the Cool Term software, stop recording.
14. Open a new window in excel and select open in the file settings.
15. Select the file you generated in CoolTerm and go to next.
16. Select commas as the delineator.
17. Open the file and resave as a csv.
18. Use the recorded data to produce plots.

The above procedure was used to test continuous stimulation with torsion. The same approach stated in the last section was used for the flexion and extension detection. Like the threshold-based torsion identification, the torsion threshold here was selected via an empirical calibration phase. Deflection classified as “torsion” was achieved when flex sensor 1 and 2 dropped below the 15.92 KΩ torsion threshold. The deflection state of torsion was characterized by continuous increases in the stimulation frequency proportional to the torsion movement.

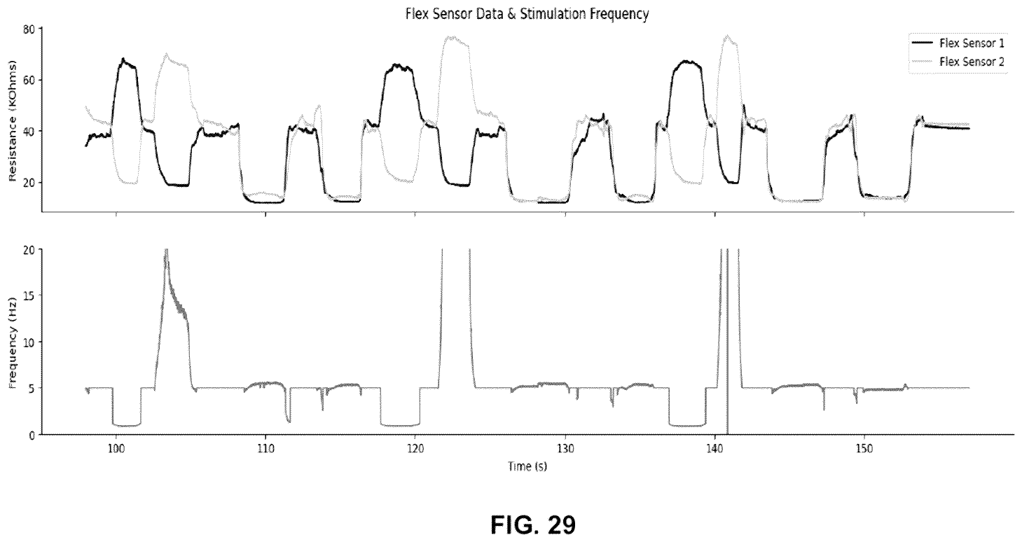

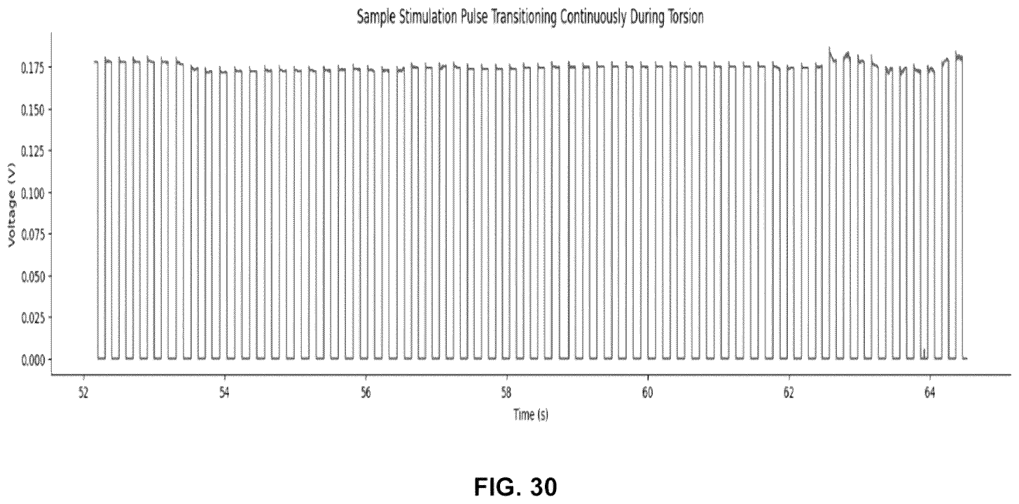

In this test, the increased mobility of the isolated flex sensors resulted in large changes in the flex sensor resistances. These changes resulted in a stimulation frequency that approached an outlying maximum. The frequency plot of FIG. 30 has a modified y-axis that focuses the plot on the relevant frequency fluctuations, especially regarding those that occur in torsion. FIG. 29 depicts flex sensor values during our testing procedure and highlighted regions to indicate the state. Flexion is indicated by dark grey highlighting, extension is shown in light grey, and torsion is highlighted in very light grey. FIG. 30 depicts a sample stimulation pulse during the two torsion states. The gray, light gray, and black lines represent the period of the pulse from several different points in the stimulation pulse. The black line shows the largest period, the light gray is the smallest period and the gray is the baseline. A size comparison of these three pulse widths is provided beneath the black line and an enlarged version is shown at the side.

### Inertial Measurement Unit

In another embodiment of the spinal cord stimulation the sensor is an inertial measurement unit (IMU). The IMU measures movement at a location outside the spinal column. In one embodiment, the IMU us attached to the head and measures the positioning and movement of the head.

Three closed loop algorithms were programed to test the efficacy of
1. A ‘threshold’ value of head orientation or movement above which the stimulation is turned up or down according to the patient's needs.
2. A classifier that detects specific ‘states’ of the head orientation or detects specific movements as they occur. The classifier is a discrete program that has a finite number of recognized states or movements, as well as programs to send to the stimulator.
3. A continuous decoder would actively monitor the timeseries data from the sensor and update stimulation on a continuous spectrum (e.g. the stimulation can be ramped from 1-5 mA depending on the amount of bend in the neck).

FIG. 31 depicts a diagram of an IMU. The following components were used in the construction of the IMU for this test.
Adafruit ESP32-S3 Feather with 2 MB PSRAM and 4 MB RAM 3151
Adafruit BNO085 3153
Stemma QT cable 3155
LED 3157
240Ω Resistor 3159

These components were wired together in accordance with the following schematic. A microcontroller was used in conjunction with an inertial measurement unit (IMU) to measure head location. The device was worn on the ear in a manner similar to that described in the extension to the provisional patent. The IMU detected 9 degrees of freedom that produce the 4 quaternion outputs used to detect the states: “left turn”, “right turn”, “up”, “down”, “left bend”, & “right bend.” The microcontroller interpreted the sensor data and changed the frequency of an output square waveform that could be used to trigger spinal stimulation. The output waveform was visualized using an LED, and digitally captured by the microcontroller for review.

FIG. 32 is a flow chart of the communication in the system. The IMU 3261 detects the movements of a subject's head. The data related to that movement is passed from the IMU to the microcontroller 3263. The microcontroller sends a stimulation output instruction to the stimulation circuit 3265. The stimulation circuit stimulates the spinal cord or an area next to the spinal cord. The stimulation circuit also sends a reading of the stimulation pattern to the microcontroller.

FIG. 33 depicts the IMU 3361 in place on the head of a test subject. It is placed on the head resting behind the user's ear. As the sensor is an inertial movement unit, it senses movement. Placing the IMU on the head provides a means of measuring the movement of the cervical spine, and assisting in movement.

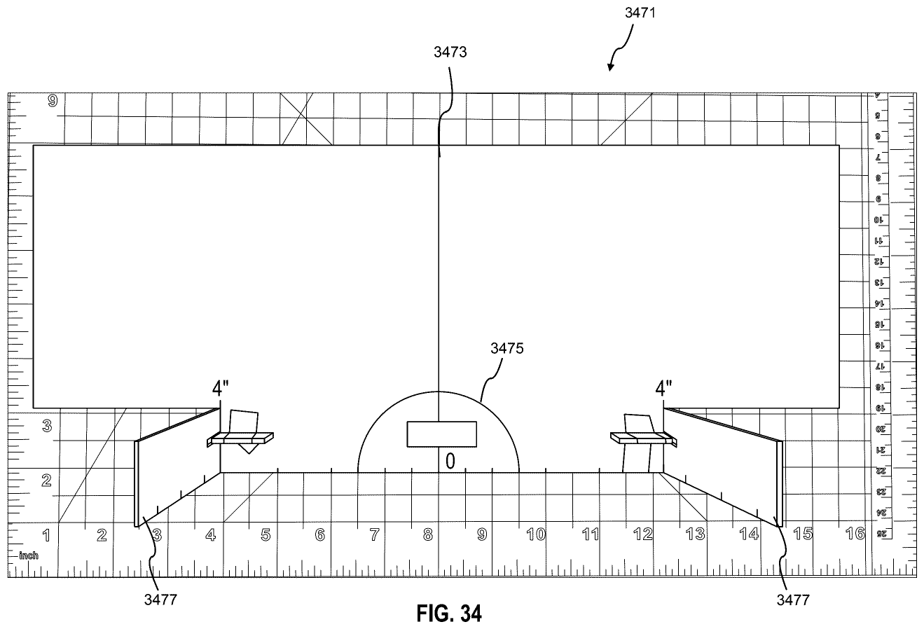

FIG. 34 depicts a motion guide 3471 for use in testing the IMU. The line in the center 3473 is the central axis that is the neutral position. The arch 3475 around this center represents the resting place for the subject's chin when used for left and right turning. The motion guiding pins are located 4″ away from the central axis, and they are provided to ensure repeatability of testing and movements.

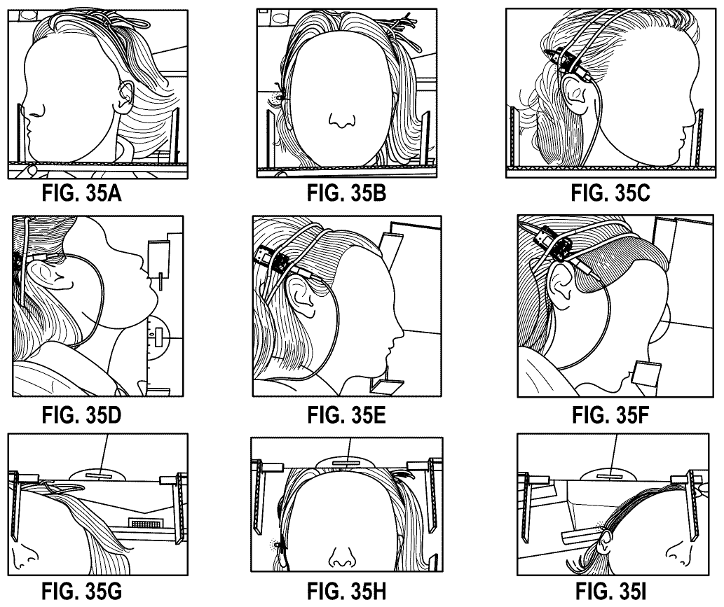

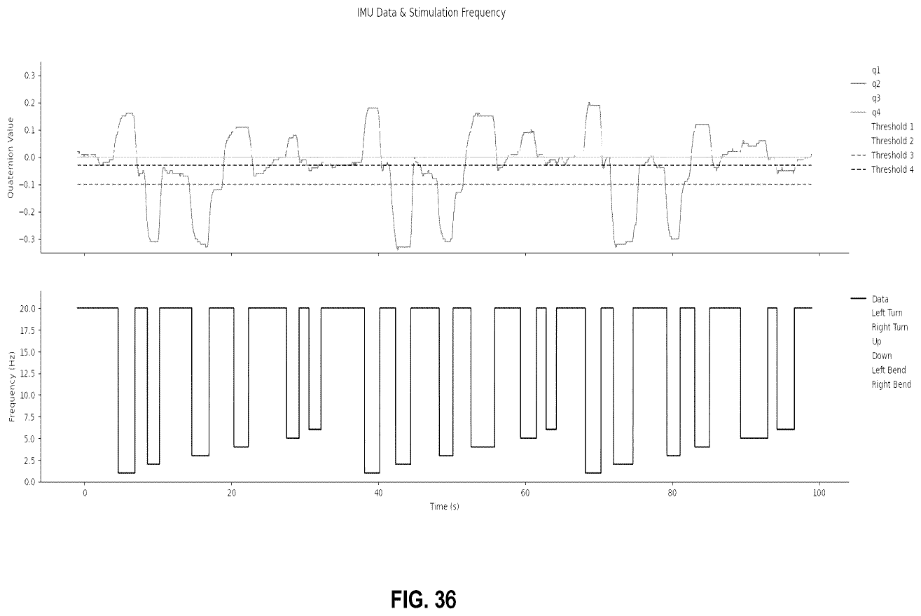

FIGS. 35A-35I depict the various head movements. A motion guide is used so that head motions can be reproduced with some accuracy. In testing turning, the motion guide is placed on a flat surface, the chin is placed on the motion guide. In testing up and down motions, the motion guide is positioned vertically so the stop pins are above or below a midpoint. When bending is measured, the motion guide is placed above, so the forehead or crown of the head is placed at a midpoint, when the neck is bent, the nose will touch the end of the stop pins. FIG. 35A shows the head turned to the left. The nose of the test subject is moved to the left guide pin. The guide pins enable reproduceable movements to test the IMU. FIG. 35B shows the head in a neutral position. FIG. 35C shows the head turned to the right. The nose of the test subject is moved to the right guide pin. FIG. 35D shows the head lifted up. The motion guide is moved to a position where the guide pins are placed so the subject's nose will touch one guide pin when lifted, and the other stop pin is placed so the nose will touch it when lowered. FIG. 35E shows the head in a neutral position. FIG. 35F shows the head in a downward position. The nose touches the lower stop pin.

### Threshold Based Stimulation Modulation

This example depicts a model of Inertial Measurement Unit sensors as they would function in use. The IMU sensors are placed on the head behind the ear, as the sensor would be placed on a patient. The test subject performed various head movements, to show the use of the sensing and stimulation components of the sensor as they work in concert in a closed loop system to modulate stimulation of the stimulation sensor in response to the sensor value of the sensor component.

Procedure:
1. Upload “threshold_state_detect.ino” to the ESP32-S3 Feather.
2. Wait for the code to compile and fully upload to the Feather, then press the reset button on the board.

3. Open the Serial Monitor in the Arduino IDE
4. Follow the prompt to place the sensor. The sensor should be situated behind the left ear and secured in place using nylon or elastic straps. Correct your posture and ensure your head is facing upright and forward from your body
5. Once the sensor is comfortably in place, navigate to the serial input field, type any letter, and press enter.
6. Visually verify that the data starts streaming and close the Arduino serial monitor.
7. Open the CoolTerm software and connect to the appropriate port with the appropriate baud rate.
8. Go to Connection and then hover over Capture to Text/Binary file.
9. Select Start from this menu and when prompted name the data file with the ending “.txt”
10. Return to the CoolTerm main window and press connect.
11. Verify that the data is now streaming to this serial monitor.
12. Obtain the cardboard motion guide.
13. Holding the cardboard motion guide beneath your chin so that the center of your face is aligned at 0.
14. Rotate your head to the left until your nose touches the pin. Hold for 3 seconds then return to the neutral or rest position.
15. Rotate your head to the right until your nose touches the pin. Hold for 3 seconds then return to the rest position.
16. Move the cardboard motion guide to the right side of your head, aligning the 0 with your eyes.
17. Rotate your head up as far as you can or until your nose touches the pin on the motion guide. Hold for 3 seconds then return to neutral position.
18. Rotate your head down as far as you can or until your nose touches the pin on the motion guide. Hold for 3 seconds then return to the neutral position.
19. Move the motion guide to the top of your forehead with the pins and measurement masks facing down. Align the 0 line with the center of your face as best as possible.
20. Tilt your head from this position towards the left as far as possible or until the tip of your nose is aligned with the tip of the pin. Hold for 3 seconds then return to the neutral position.
21. Tilt your head to the right as far as possible or until the tip of your nose reaches the motion guide pin. Hold this position for 3 seconds before returning to the neutral position.
22. Repeat steps 13-21 at least three times.
23. In the CoolTerm software go to the connection menu and under capture test to binary/text file, select stop.
24. Disconnect from the serial terminal.
25. Open excel.
26. Go to open in excel and select the file where you saved your data.
27. Select commas as the delimiter.
28. Open your file.
29. Save as a csv.

The data in table 1 was collected in accordance with the above-described testing procedure. IMU quaternion data was used for head state classification in these tests. The thresholds for quaternion (q) values were determined empirically based on a calibration phase where the device was worn to observe the resulting q changes. Thresholds were selected to represent a moderate amount of head movement, representative of the level of movement expected to yield user-perceived differences in electrical stimulation. Thresholds 1 & 3 are inverses at q=±0.1. The remaining thresholds, 2 & 4, are also inverses at q=±0.02. State identification occurs according to the conditions detailed in Table 1.

TABLE 1

| | q1 | q2 | q3 | q4 |
|---|---|---|---|---|
| Left Turn | <threshold 3 | >threshold 1 | ~ | ~ |
| | (−0.1) | (0.1) | | |
| Right Turn | >threshold 1 | <threshold 3 | >threshold 2 | ~ |
| | (0.1) | (−0.1) | (0.02) | |
| Up | <threshold 3 | <threshold 3 | >threshold 2 | ~ |
| | (−0.1) | (−0.1) | (0.02) | |
| Down | >threshold 1 | >threshold 1 | ~ | ~ |
| | (0.1) | (0.1) | | |
| Left Bend | ~ | ~ | >threshold 1 | ~ |
| | | | (0.02) | |
| Right Bend | ~ | ~ | <threshold 3 | ~ |
| | | | (−0.02) | |

The stimulation frequency of each state is shown in Table 2. Low frequencies of 1-6 Hz were chosen to make it easier to observe the frequency changes in the timeseries plots below, but any stimulation parameter can be set using the state decoder described here, including higher frequencies such as those used therapeutically in our system (40-100 Hz).

TABLE 2

| State | Stimulation Frequency (Hz) |
|---|---|
| Rest | 20 |
| Left Turn | 1 |
| Right Turn | 2 |
| Up | 3 |
| Down | 4 |
| Left Bend | 5 |
| Right Bend | 6 |

FIG. 36 depicts the IMU quaternion data. In this plot, the thresholds are represented by dashed lines of varying shades of grey. The grey shading regions are used to indicate the state of motion which can be determined using the legend to the right.

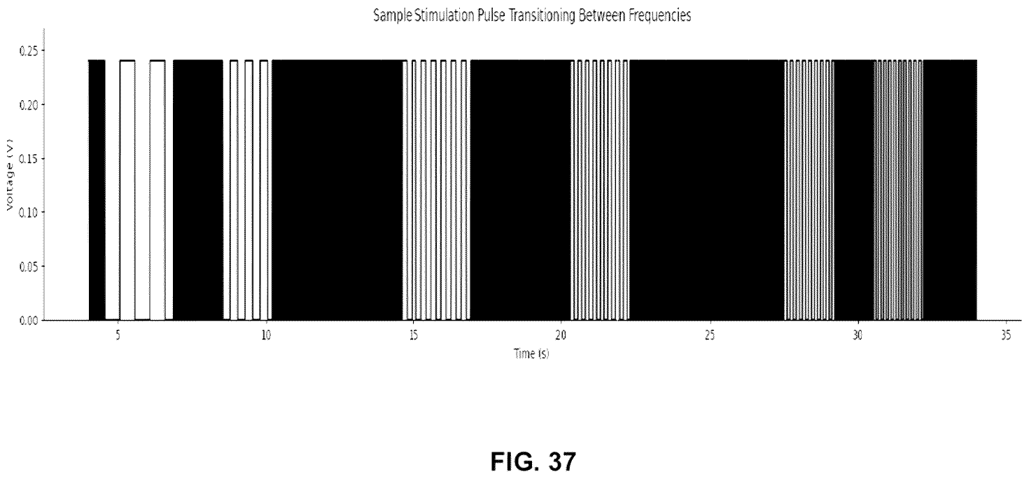

FIG. 37 depicts the stimulation pulse throughout a 30 second window of the experiment. In this figure you can see the frequency go from rest to the new state determined frequency.

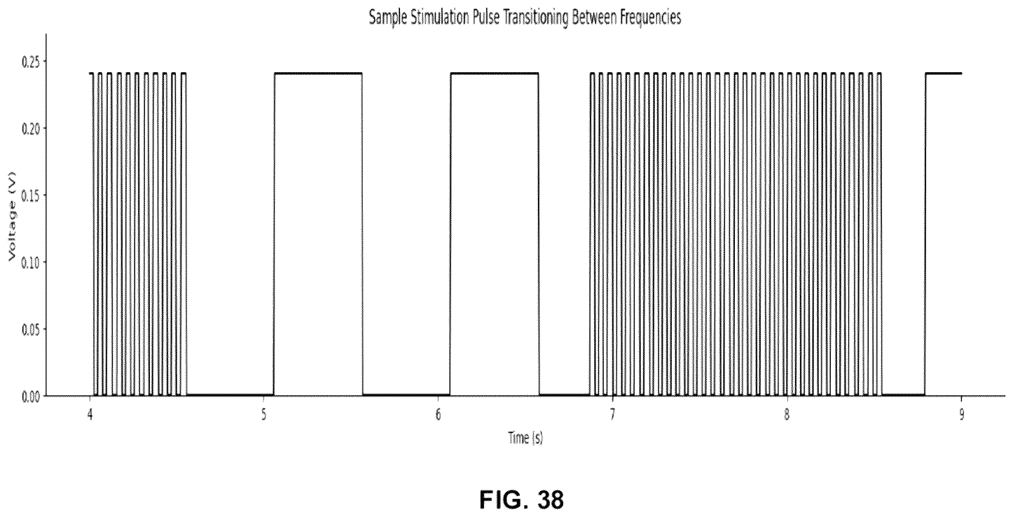

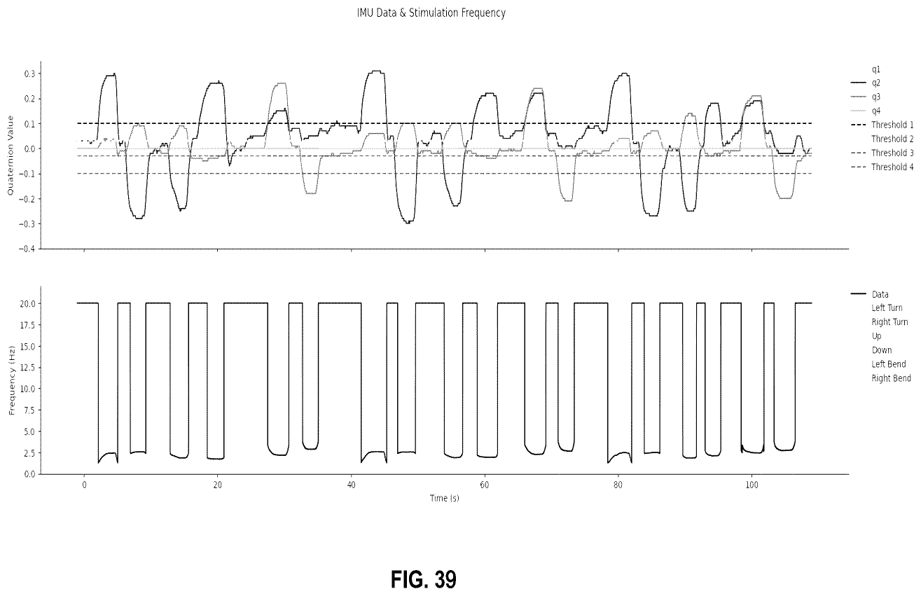

FIG. 38 depicts a zoomed in section of FIG. 6 to shown that the dense black regions of that plot are high frequency stimulation pulses.

These tests and the associated figures show the efficacy of a simple threshold-based head state detection algorithm for an ear worn IMU sensor. They also capture the stimulation frequency changes that can be implemented in stimulation as a result of the state detected.

### Continuous Movement Proportional Stimulation Modulation

This example depicts a model of Inertial Measurement Unit sensors as they would function in use, with the addition of continuous stimulation. The continuous stimulation assists in reaching the stimulation threshold for movement. The raised level of baseline stimulation lowers the amount of stimulation needed to reach the movement threshold. The IMU sensors are placed on the head behind the ear, as the sensor would be placed on a patient. The test subject performed various head movements, to show the use of the sensing and stimulation components of the sensor as they work in concert in a closed loop system to modulate stimulation of the stimulation sensor in response to the sensor value of the sensor component.

Procedure:
1. Upload “cont_state_detect.ino” to the ESP32-S3 Feather.
2. Wait for the code to compile and fully upload to the Feather, then press the reset button on the board.
3. Open the Serial Monitor in the Arduino IDE
4. Follow the prompt to place the sensor. The sensor should be situated behind the left ear and secured in place using nylon or elastic straps. Correct your posture and ensure your head is facing upright and forward from your body
5. Once the sensor is comfortably in place, navigate to the serial input field, type any letter, and press enter.
6. Visually verify that the data starts streaming and close the Arduino serial monitor.
7. Open the CoolTerm software and connect to the appropriate port with the appropriate baud rate.
8. Go to Connection and then hover over Capture to Text/Binary file.
9. Select Start from this menu and when prompted name the data file with the ending “.txt”
10. Return to the CoolTerm main window and press connect.
11. Verify that the data is now streaming to this serial monitor.
12. Obtain the cardboard motion guide.
13. Holding the cardboard motion guide beneath your chin so that the center of your face is aligned at 0.
14. Rotate your head to the left until your nose touches the pin. Hold for 3 seconds then return to the neutral or rest position.
15. Rotate your head to the right until your nose touches the pin. Hold for 3 seconds then return to the rest position.
16. Move the cardboard motion guide to the right side of your head, aligning the 0 with your eyes.
17. Rotate your head up as far as you can or until your nose touches the pin on the motion guide. Hold for 3 seconds then return to neutral position.
18. Rotate your head down as far as you can or until your nose touches the pin on the motion guide. Hold for 3 seconds then return to the neutral position.
19. Move the motion guide to the top of your forehead with the pins and measurement masks facing down. Align the 0 line with the center of your face as best as possible.
20. Tilt your head from this position towards the left as far as possible or until the tip of your nose is aligned with the tip of the pin. Hold for 3 seconds then return to the neutral position.
21. Tilt your head to the right as far as possible or until the tip of your nose reaches the motion guide pin. Hold this position for 3 seconds before returning to the neutral position.
22. Repeat steps 13-21 at least three times.
23. In the CoolTerm software go to the connection menu and under capture test to binary/text file, select stop.
24. Disconnect from the serial terminal.
25. Open excel.
26. Go to open in excel and select the file where you saved your data.
27. Select commas as the delimiter.
28. Open your file.
29. Save as a csv.

The same thresholds described above in the threshold-based section were used in this program. The table 3 shows the state identification conditions. Once the device was in one of the motion states, the stimulation frequency was modulated continuously and proportionally to the larger q value. It is obvious that other, more complex algorithms could be implemented which integrate all 4 quaternion values, and change stimulation parameters based on this combined metric.

TABLE 3

| | q1 | q2 | q3 | q4 |
|---|---|---|---|---|
| Left Turn | <threshold 3 | >threshold 1 | ~ | ~ |
| | (−0.1) | (0.1) | | |
| Right Turn | >threshold 1 | <threshold 3 | >threshold 2 | ~ |
| | (0.1) | (−0.1) | (0.02) | |
| Up | <threshold 3 | <threshold 3 | ~ | ~ |
| | (−0.1) | (−0.1) | | |
| Down | >threshold 1 | >threshold 1 | ~ | ~ |
| | (0.1) | (0.1) | | |
| Left Bend | ~ | ~ | >threshold 1 | ~ |
| | | | (0.1) | |
| Right Bend | ~ | ~ | <threshold 3 | ~ |
| | | | (−0.1) | |

The stimulation base frequency of each state is shown table 4 below with the way that the frequency changed in response to movement.

TABLE 4

| | Stimulation | Stimulation |
|---|---|---|
| State | Frequency (Hz) | Frequency Change |
| Rest | 20 | |
| Left Turn | 1 | + |
| Right Turn | 2 | + |
| Up | 3 | − |
| Down | 4 | − |
| Left Bend | 5 | − |
| Right Bend | 6 | − |

FIG. 39 depicts the IMU quaternion data as it changes throughout the trail. In this plot, the thresholds are represented by dashed lines of varying shades of grey. The grey shading regions are used to indicate the state of motion which can be determined using the legend ta the right. The frequency of the stimulation pulse is shown in the plot at the bottom. The arches in this plot indicate continuous modulation of stimulation frequency once a motion state is detected.

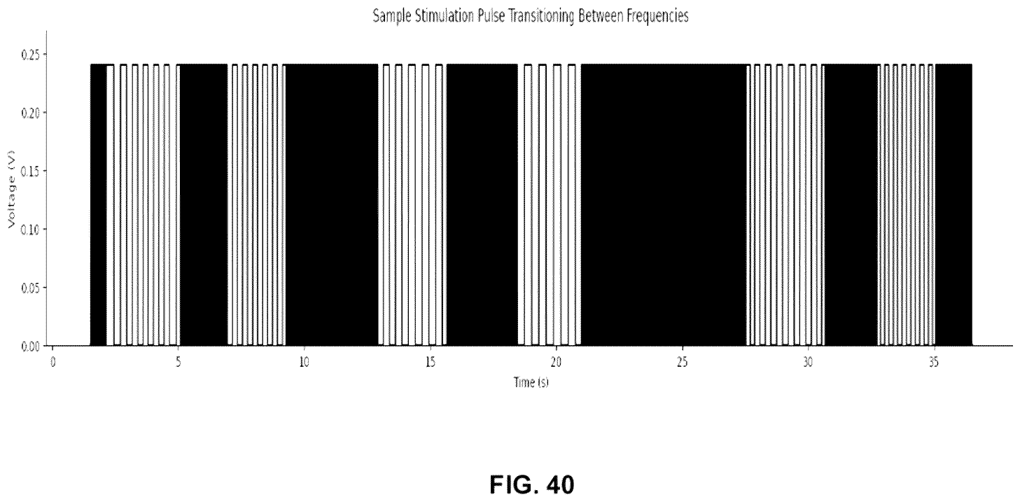

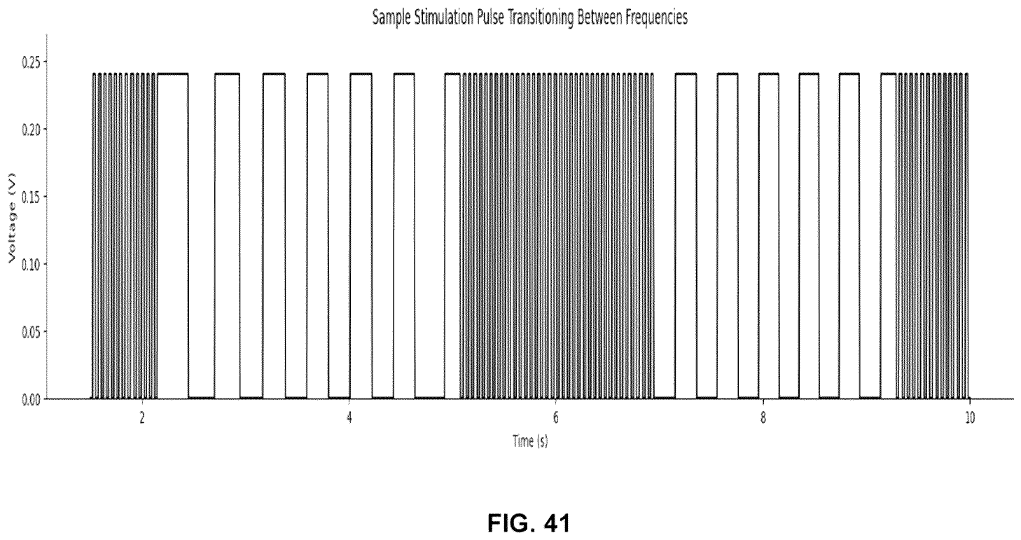

FIG. 40 shows the stimulation pulse throughout a 30 second window of the experiment. In this figure you can see the frequency go from rest to the new state determined frequency.

FIG. 41 depicts a zoomed in section from FIG. 9 to shown that the dense black regions of that plot are high frequency stimulation pulses. Modulation in stimulation frequency is indicated by changes in the period of these waveforms. A comparison of the periods is provided by the overlaid arrows.

This algorithm depicts a continuous stimulation adjustment algorithm. This implementation builds off the threshold-based algorithm to detect states, and continuously change stimulation proportionally to head movement once the state is detected (Objective algorithm 2). Additionally, this data also supports Objective algorithm 3 in showing the ability to continuously change the stimulation frequency. While this algorithm is written to only begin continuously updating the stimulation parameters after an initial threshold of movement is detected in a given direction, it is clear that an algorithm could be written to continuously control stimulation parameters based on all head movements without first needing to detect a threshold.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

### Exemplary Embodiments

Embodiment 1 is, a method of detecting movement of a spine, and stimulating specific regions of the spinal cord, the method comprising:
providing a sensor having one or more sensing elements to sense movement of the spine;
providing one or more stimulating elements configured to electrically stimulate an area proximate the spine;
detecting, via the sensor, a movement of the spine;
and
modulating an electrical stimulus provided by the one or more stimulating elements at an area proximate the spine in response to the detected movement.

Embodiment 2 is the method of embodiment 1, wherein the one or more sensing elements each include a first set of sensing elements and a second set of sensing elements.

Embodiment 3 is the method of embodiment 2, wherein the first set of sensing elements is substantially aligned with the second set of sensing elements when the sensor is in the initial state.

Embodiment 4 is the method of embodiment 2, wherein the first set of sensing elements is offset from the second set of sensing elements when the sensor detects movement.

Embodiment 5 is the method of embodiment 2, wherein the first set of sensing elements and the second set of sensing elements each include substantially rectangular shaped sensing elements.

Embodiment 6 is the method of embodiment 2, wherein the first set of sensing elements and the second set of sensing elements each include substantially arrow-head shaped sensing elements.

Embodiment 7 is the method of embodiment 2, wherein the first set of sensing elements and the second set of sensing elements each include substantially parallelogram shaped sensing elements.

Embodiment 8 is the method of embodiment 1, wherein the sensor includes a resistive layer disposed between the substrate and the one or more sensing elements.

Embodiment 9 is the method of embodiment 8, wherein the resistive layer is formed via deposition of conductive ink on the substrate.

Embodiment 10 is the method of embodiment 8, wherein the resistive layer is formed via embedding conductive ink or particles within a silicone.

Embodiment 11 is the method of embodiment 8, wherein the sensor includes overmolding disposed on the resistive layer, the one or more sensing elements being disposed within the overmolding.

Embodiment 12 is the method of embodiment 1 wherein detecting movement comprises:
comparing an altered property value to a threshold value;
determining that the altered property value exceeds the threshold value;
calculating a difference between the altered property value and the threshold value in response to the determination that the altered property value exceeds the threshold value; and
based on the difference, calculating a spinal deviation, wherein the spinal deviation includes one or more of flexing or twisting of the spine from an initial spinal position to a deviated spinal position.

Embodiment 13 is the method of embodiment 12, wherein the spinal deviation is due to flexing of one or more muscle located proximate to the spine.

Embodiment 14 is the method of embodiment 1, wherein the one or more stimulating elements and the one or more sensing elements are the same.

Embodiment 15 is the method of embodiment 14, wherein when more than one sensing element is utilized in the sensor, the one or more stimulating elements are located on one of the sensing elements.

Embodiment 16 is the method of embodiment 14, wherein the sensor provides cervical spinal cord stimulation for treatment of neurological disease, disorders, or injuries.

Embodiment 17 is the method of embodiment 1 wherein detecting movement of the spine comprises:
measuring an initial property value;
continuously measuring an altered property value;
calculating a difference between the altered property value and the initial property value; and
based on the difference, continuously calculating a spinal deviation, wherein the spinal deviation includes one or more of flexing or twisting of the spine from an initial spinal position to a deviated spinal position.

Embodiment 18 is the method of embodiment 1, wherein the movement of the spine is due to flexing of one or more muscle and/or skeletal structures located proximate to the spine.

Embodiment 19 is the method of embodiment 1, wherein the substrate is a flexible substrate.

Embodiment 20 is the method of embodiment 12, wherein the initial property value and the altered property value are different.

Embodiment 21 is the method of embodiment 12, wherein the initial property value is an initial resistance value, and the altered property value is an altered resistance value, the initial resistance value and the altered resistance value associated with a resistance of the sensor.

Embodiment 22 is the method of embodiment 12, wherein the initial property value is an initial capacitance value, and the altered property value is an altered capacitance value, the initial capacitance value and the altered capacitance value associated with a capacitance of the sensor.

Embodiment 23 is the method of embodiment 1 further comprising:
arranging the one or more sensing elements such that the one or more sensing elements align with a direction of the deformation of the sensor.

Embodiment 24 is the method of embodiment 1, wherein one or more gaps are disposed between each of the one or more sensing elements.

Embodiment 25 is the method of embodiment 1, wherein at least one of the one or more sensing elements is substantially rectangular shaped.

Embodiment 26 is the method of embodiment 1, wherein at least one of the one or more sensing elements is substantially diamond shaped.

Embodiment 27 is the method of embodiment 1, wherein at least one of the one or more sensing elements is substantially parallelogram shaped.

Embodiment 28 is the method of embodiment 1, wherein at least one of the one or more sensing elements is substantially bowtie shaped.

Embodiment 29 is the method of embodiment 1, wherein the movement of the spine includes torsion or flexion of the spine.

Embodiment 30 is the method of embodiment 1, wherein the sensor includes a stabilizing flap configured to prevent lateral movement of the sensor relative to the spine.

Embodiment 31 is a sensor and stimulator comprising:
a sensor body and a stimulator body;
the stimulator body having a first end and a second end opposite the first end, the stimulator body configured to be placed proximate to a spine of a patient;
a plurality of sensing elements disposed on the sensor body;
one or more stimulating elements configured to electrically stimulate an area proximate the spine; and
a control circuit disposed proximate the second end of the stimulator body, wherein the control circuit is configured to:
measure an initial property associated with the sensor being in an initial state;
detect change from the initial property;
measure, upon detecting the change, an altered property associated with the sensor being in an altered state; and
correlate the altered property associated with the sensor to movement of the spine of the patient
modulate the electrical stimulus provided by the one or more stimulating elements at an area proximate the spine responsive to the altered property.

Embodiment 32 is the sensor of claim 31, wherein the initial property value and the altered property value are resistance values associated with a resistance of the sensor.

Embodiment 33 is the sensor of embodiment 31, wherein sensor body and the stimulator body are the same.

Embodiment 34 is the sensor of embodiment 31 further comprising:
a stabilizing flap extending from the body of the sensor between the first end and the second end, the stabilizing flap configured to prevent lateral movement of the sensor relative to the spine.

Embodiment 35 is the sensor of embodiment 31 further comprising:
a plurality of gaps, each disposed between the plurality of sensing elements.

Embodiment 36 is an electrode comprising:
a body having a first end and a second end opposite the first end, the body configured to placed adjacent a spine of a patient;
a plurality of stimulating elements configured to provide electrical stimulation to an area proximate the spine of the patient; and
a stabilizing flap extending from the body of the electrode between the first end and the second end, the stabilizing flap configured to prevent lateral movement of the electrode relative to the spine of the patient when the electrode is placed adjacent the spine of the patient.

Embodiment 37 is the electrode of embodiment 36, wherein the stabilizing flap includes a first lateral end and a second lateral end, the first lateral end coupling the stabilizing flap to the body and being opposite the second lateral end.

Embodiment 38 is the electrode of embodiment 37, wherein the stabilizing flap tapers in thickness from the first lateral end to the second lateral end.

Embodiment 39 is the electrode of embodiment 37, wherein the stabilizing flap tapers in thickness from the first lateral end to an area between the first lateral end and the second lateral end.

Embodiment 40 is the electrode of embodiment 37, wherein the plurality of stimulating elements are disposed on the stabilizing flap proximate the second lateral end.

Embodiment 41 is the electrode of embodiment 37, wherein the plurality of stimulating elements are disposed on the stabilizing flap proximate the first lateral end.

Embodiment 42 is the electrode of embodiment 37, wherein the plurality of stimulating elements are disposed on the stabilizing flap embodiment the first lateral end and proximate the second lateral end.

Embodiment 43 is the electrode of embodiment 36, wherein the electrode includes a resistive layer disposed between the body and the plurality of stimulating elements.

Embodiment 44 is the electrode of embodiment 43, wherein the electrode includes overmolding disposed on the resistive layer, the plurality of stimulating elements being disposed within the overmolding.

Embodiment 45 is the electrode of embodiment 44, wherein the overmolding extends from the body to form the stabilizing flap.

Embodiment 46 is the electrode of embodiment 36, wherein the body includes a first side and a second side, and the stabilizing flap extends from one of the first side and the second side.

Embodiment 47 is the electrode of embodiment 46, wherein stabilizing flap has a thickness that is substantially the same as a thickness of the body and the plurality of stimulating elements are disposed on a single side of the electrode proximate one of the first side and the second side of the body.

Embodiment 48 is the electrode of embodiment 46, wherein the stabilizing flap extends from both the first side and the second side.

Embodiment 49 is the electrode of embodiment 36, wherein the body is substantially linear.

Embodiment 50 is the electrode of embodiment 36, wherein the plurality of stimulating elements are disposed along a length of the body between the first end and the second end.

Embodiment 51 is the electrode of embodiment 36 further comprising:
a second body having a plurality of second stimulating elements, wherein the stabilizing flap is disposed between the body and the second body.

Embodiment 52 is the electrode of embodiment 36, wherein the stabilizing flap has a length lesser than or equal to the length of the body.

Embodiment 53 is the electrode of embodiment 36, wherein the stabilizing flap is comprised of a biocompatible and flexible material.

Embodiment 54 is the electrode of embodiment 36, wherein the stabilizing flap extends from a single side of the body.

Embodiment 55 is the electrode of embodiment 36, wherein the electrode is configured to be disposed within an epidural space of the patient.

Embodiment 55 is the electrode of embodiment 36, wherein the electrode is disposed within the patient lateral to the spine and proximate a dorsal root of the patient, and the stabilizing flap is configured to extend over the spine.

Embodiment 57 is the electrode of embodiment 36, wherein the stabilizing flap is configured to extend through a mid-sagittal plane of the patient when the electrode is disposed within the patient.

Embodiment 58 is the electrode of embodiment 36, wherein the stabilizing flap is substantially rectangular shaped.

Embodiment 59 is the electrode of claim 36, wherein the stabilizing flap has a maximum thickness less than a maximum thickness of the body.

Embodiment 60 is the An electrode comprising:
a body having a first end and a second end opposite the first end, the body configured to be placed adjacent a spine of a patient;
a plurality of sensing elements disposed between the first end and the second end;
a control circuit coupled to at least one of the plurality of sensing elements, the control circuit configured to detect deformation of the body; and
a stabilizing flap extending from the body of the electrode between the first end and the second end, the stabilizing flap configured to prevent lateral movement of the electrode relative to the spine of the patient when the electrode is placed adjacent the spine of the patient.

Embodiment 61 is the electrode of embodiment 60 further comprising:
a plurality of stimulating elements configured to provide stimulation to an area proximate the spine of the patient.

Embodiment 62 is the method of embodiment 61, wherein the electrode provides cervical spinal cord stimulation for treatment of neurological disease, disorders, or injuries.

Embodiment 63 is the electrode of embodiment 61, wherein the plurality of stimulating elements is different than the plurality of sensing elements.

Embodiment 64 is the electrode of embodiment 60 further comprising: a plurality of gaps disposed between the plurality of sensing elements.

Embodiment 65 is a sensor for detecting movement of the spine, the sensor comprising:
a body having a first end and a second end opposite the first end, the body configured to be placed adjacent a spine of a patient;
a plurality of sensing elements and a plurality of stimulating elements disposed between the first end and the second end, the plurality of stimulating elements configured to provide stimulation to an area proximate the spine of the patient; and
a control circuit coupled to at least one of the plurality of sensing elements and at least one of the plurality of stimulating elements, the control circuit configured to detect deformation of the body and control the level of the stimulation

Embodiment 66 is the sensor of embodiment 66 further comprising:
a stabilizing flap extending from the body of the electrode between the first end and the second end, the stabilizing flap configured to prevent lateral movement of the electrode relative

1-66. (canceled)

67. A method of detecting movement of a spine, and stimulating specific regions of the spinal cord, the method comprising:
providing a sensor having one or more sensing elements to sense movement of the spine;
providing one or more stimulating elements configured to electrically stimulate an area proximate the spine;
detecting, via the sensor, a movement of the spine;
modulating an electrical stimulus provided by the one or more stimulating elements at an area proximate the spine in response to the detected movement; and
arranging the one or more sensing elements such that the one or more sensing elements align with a direction of deformation of the sensor.

68. The method of claim 67, wherein the one or more sensing elements each include a first set of sensing elements and a second set of sensing elements.

69. The method of claim 68, wherein the first set of sensing elements is substantially aligned with the second set of sensing elements when the sensor is in the initial state.

70. The method of claim 68, wherein the first set of sensing elements is offset from the second set of sensing elements when the sensor detects movement.

71. The method of claim 67, wherein the sensor includes a resistive layer disposed between a substrate and the one or more sensing elements.

72. The method of claim 71, wherein the substrate is a flexible substrate.

73. The method of claim 71, wherein the resistive layer is formed via deposition of conductive ink on the substrate.

74. The method of claim 71, wherein the resistive layer is formed via embedding conductive ink or particles within a silicone.

75. The method of claim 71, wherein the sensor includes overmolding disposed on the resistive layer, the one or more sensing elements being disposed within the overmolding.

76. The method of claim 67, wherein detecting movement comprises:
comparing an altered property value to a threshold value;
determining that the altered property value exceeds the threshold value;
calculating a difference between the altered property value and the threshold value in response to the determination that the altered property value exceeds the threshold value; and
based on the difference, calculating a spinal deviation, wherein the spinal deviation includes one or more of flexing or twisting of the spine from an initial spinal position to a deviated spinal position.

77. The method of claim 76, wherein the deviated spinal position is due to flexing of one or more muscle located proximate to the spine.

78. The method of claim 76, wherein the stimulating elements provide cervical spinal cord stimulation for treatment of neurological disease, disorders, or injuries.

79. The method of claim 67, wherein detecting movement of the spine comprises:
measuring an initial property value;
continuously measuring an altered property value;
calculating a difference between the altered property value and the initial property value; and
based on the difference, continuously calculating a spinal deviation, wherein the spinal deviation includes one or more of flexing or twisting of the spine from an initial spinal position to a deviated spinal position.

80. The method of claim 79, wherein the movement of the spine is due to flexing of one or more muscle and/or skeletal structures located proximate to the spine.

81. The method of claim 79, wherein the initial property value and the altered property value are different.

82. The method of claim 79, wherein the initial property value is an initial resistance value, and the altered property value is an altered resistance value, the initial resistance value and the altered resistance value associated with a resistance of the sensor.

83. The method of claim 79, wherein the initial property value is an initial capacitance value, and the altered property value is an altered capacitance value, the initial capacitance value and the altered capacitance value associated with a capacitance of the sensor.

84. The method of claim 67, wherein one or more gaps are disposed between each of the one or more sensing elements.

85. The method of claim 67, wherein the movement of the spine includes torsion or flexion of the spine.
